(19) United States
(12) Patent Application Publication
(10) Pub. No.: US 2023/0416318 A1
(43) Pub. Date: Dec. 28, 2023

(54) OSTEOANABOLISM BY 14-3-3ZETA

(71) Applicant: The University of Toledo, Toledo, OH (US)

(72) Inventors: Ritu Chakravarti, Toledo, OH (US); Saurabh Chattopadhyay, Toledo, OH (US)

(73) Assignee: The University of Toledo, Toledo, OH (US)

(21) Appl. No.: 18/314,240

(22) Filed: May 9, 2023

Related U.S. Application Data
(60) Provisional application No. 63/340,087, filed on May 10, 2022.

(51) Int. Cl. C07K 14/47; A61P 19/08

(52) U.S. Cl. CPC C07K 14/4702; A61P 19/08

(57) ABSTRACT

Methods for promoting bone growth, preventing bone damage, and regulating collagen production, involving the administration of a 14-3-3zeta protein or 14-3-3zeta mRNA, are described.

### RELATED APPLICATIONS

This application claims priority to U.S. Provisional Application No. 63/340,087 filed under 35 U.S.C. § 111(b) on May 10, 2022, the disclosure of which is incorporated herein by reference in its entirety.

### STATEMENT REGARDING FEDERALLY SPONSORED RESEARCH

This invention was made with no government support. The government has no rights in this invention.

### SEQUENCE LISTING

The instant application contains a Sequence Listing which has been submitted electronically in XML format and is hereby incorporated by reference in its entirety. Said XML copy, created on May 4, 2023, is named 63151-US-NP_D2021-48_SL.xml and is 16,042 bytes in size.

### BACKGROUND

Inflammatory arthritis (IA) is a common disease that affects millions of individuals worldwide, more than 1% of the world population. Proinflammatory events during IA pathogenesis are well studied; however, loss of protective immunity remains underexplored. Natural host-protective immune responses to suppress, or prophylactic immunization to prevent, IA remain unknown. There are currently no preventive treatments for fractures/osteoarthritis.

Bone loss is associated with normal aging and clinical pathologies, including osteoporosis, arthritis, and chemotherapy. Fractures alone cost $14 billion per year in the US. While several drugs targeting the prevention of bone loss have been in clinical use (namely, Romosozumab, Abaloparatide, and Teraparatide), there is limited knowledge of drugs that promote bone growth. Furthermore, these known drugs are biological treatments that need to be injected regularly, last a short time, and need to be combined with additional anti-resorptive drugs. Thus, there is a need in the art for new drugs that promote bone growth. Developing a drug with an osteo-anabolic effect would be highly desirable for prophylactic and therapeutic treatments for aged and immunosuppressed individuals.

### SUMMARY

Provided is a method of promoting bone growth in a subject, the method comprising administering to a subject an effective amount of a 14-3-3zeta protein or 14-3-3zeta mRNA to promote bone growth in the subject. In certain embodiments, the subject has an elevated risk of a bone fracture. In certain embodiments, the subject is at least 55 years old, is a post-menopausal woman, has osteoporosis, has diabetes, is trauma-affected, or is post-operative.

In certain embodiments, the effective amount is about 1 mg/kg.

In certain embodiments, the 14-3-3zeta protein is in a composition with an adjuvant comprising incomplete Freund's adjuvant (IFA). In certain embodiments, the 14-3-3zeta mRNA is in a composition with an adjuvant comprising incomplete Freund's adjuvant (IFA).

In certain embodiments, the method comprises administering two doses of the 14-3-3zeta protein or 14-3-3zeta mRNA to the subject. In particular embodiments, the two doses promotes bone growth in the subject for at least 45 days.

In certain embodiments, the subject has inflammatory arthritis, and administration of the 14-3-3zeta protein or 14-3-3zeta mRNA causes a reduction of inflammatory arthritis symptoms in the subject.

In certain embodiments, the 14-3-3zeta protein or 14-3-3zeta mRNA reduces Illb expression in the subject. In certain embodiments, the 14-3-3zeta protein or 14-3-3zeta mRNA increases Illrn expression in the subject. In certain embodiments, the 14-3-3zeta protein or 14-3-3zeta mRNA reduces circulating IL-1β protein in the subject. In certain embodiments, the 14-3-3zeta protein or 14-3-3zeta mRNA reduces expression of one or more proinflammatory cytokines in the subject selected from the group consisting of Cxcl1, Ifng, and Tnfa. In certain embodiments, the 14-3-3zeta protein or 14-3-3zeta mRNA suppresses RANKL-stimulated signal transduction in the subject.

In certain embodiments, the 14-3-3zeta protein is administered to the subject through a dermal patch. In certain embodiments, the 14-3-3zeta protein is administered to the subject through an ointment. In certain embodiments, the 14-3-3zeta protein or the 14-3-3zeta mRNA is administered to the subject through an injectable vaccine.

Further provided is a method of preventing bone damage in a subject, the method comprising administering to the subject an effective amount of a 14-3-3zeta protein or 14-3-3zeta mRNA to prevent bone damage in the subject. In certain embodiments, the subject has an elevated risk of a bone fracture. In certain embodiments, the subject is at least 55 years old, is a post-menopausal woman, has osteoporosis, has diabetes, is trauma-affected, or is post-operative.

In certain embodiments, the effective amount is about 1 mg/kg.

In certain embodiments, the 14-3-3zeta protein is in a composition with an adjuvant comprising incomplete Freund's adjuvant (IFA). In certain embodiments, the 14-3-3zeta mRNA is in a composition with an adjuvant comprising incomplete Freund's adjuvant (IFA).

In certain embodiments, the method comprises administering two doses of the 14-3-3zeta protein or 14-3-3zeta mRNA to the subject.

In certain embodiments, the subject has inflammatory arthritis, and administration of the 14-3-3zeta protein or 14-3-3zeta mRNA causes a reduction of inflammatory arthritis symptoms in the subject.

In certain embodiments, the 14-3-3zeta protein or 14-3-3zeta mRNA reduces Illb expression in the subject. In certain embodiments, the 14-3-3zeta protein or 14-3-3zeta mRNA increases Illrn expression in the subject. In certain embodiments, the 14-3-3zeta protein or 14-3-3zeta mRNA reduces circulating IL-1β protein in the subject. In certain embodiments, the 14-3-3zeta protein or 14-3-3zeta mRNA reduces expression of one or more proinflammatory cytokines in the subject selected from the group consisting of Cxcl1, Ifng, and Tnfa. In certain embodiments, the 14-3-3zeta protein or 14-3-3zeta mRNA suppresses RANKL-stimulated signal transduction in the subject.

In certain embodiments, the 14-3-3zeta protein is administered to the subject through a dermal patch. In certain embodiments, the 14-3-3zeta protein is administered to the subject through an ointment. In certain embodiments, the 14-3-3zeta protein or the 14-3-3zeta mRNA is administered to the subject through an injectable vaccine.

Further provided is a method of regulating collagen production in a subject, the method comprising administering to the subject an effective amount of a 14-3-3zeta protein or 14-3-3zeta mRNA to regulate collagen production in the subject. In certain embodiments, the subject has an elevated risk of a bone fracture. In certain embodiments, the subject is at least 55 years old, is a post-menopausal woman, has osteoporosis, has diabetes, is trauma-affected, or is post-operative.

In certain embodiments, the effective amount is about 1 mg/kg.

In certain embodiments, the 14-3-3zeta protein is in a composition with an adjuvant comprising incomplete Freund's adjuvant (IFA). In certain embodiments, the 14-3-3zeta mRNA is in a composition with an adjuvant comprising incomplete Freund's adjuvant (IFA).

In certain embodiments, the method comprises administering two doses of the 14-3-3zeta protein or 14-3-3zeta mRNA to the subject.

In certain embodiments, the subject has inflammatory arthritis, and administration of the 14-3-3zeta protein or 14-3-3zeta mRNA causes a reduction of inflammatory arthritis symptoms in the subject.

In certain embodiments, the 14-3-3zeta protein or 14-3-3zeta mRNA reduces Illb expression in the subject. In certain embodiments, the 14-3-3zeta protein or 14-3-3zeta mRNA increases Illrn expression in the subject. In certain embodiments, the 14-3-3zeta protein or 14-3-3zeta mRNA reduces circulating IL-1β protein in the subject. In certain embodiments, the 14-3-3zeta protein or 14-3-3zeta mRNA reduces expression of one or more proinflammatory cytokines in the subject selected from the group consisting of Cxcl1, Ifng, and Tnfa. In certain embodiments, the 14-3-3zeta protein or 14-3-3zeta mRNA suppresses RANKL-stimulated signal transduction in the subject.

In certain embodiments, the 14-3-3zeta protein is administered to the subject through a dermal patch. In certain embodiments, the 14-3-3zeta protein is administered to the subject through an ointment. In certain embodiments, the 14-3-3zeta protein or the 14-3-3zeta mRNA is administered to the subject through an injectable vaccine

Further provided is a transdermal patch comprising a composition comprising a protein, wherein the transdermal patch is configured to release the protein over time, and wherein the protein has at least 95% sequence identity to 14-3-3zeta. In certain embodiments, the protein has at least 98% sequence identity to 14-3-3zeta. In certain embodiments, the protein is 14-3-3zeta.

Further provided is a pharmaceutical composition comprising a protein having at least 95% sequence identity to 14-3-3zeta, or a 14-3-3zeta mRNA; and a pharmaceutically acceptable carrier, diluent, or adjuvant; wherein the pharmaceutical composition is in the form of an injectable vaccine. In certain embodiments, the protein has at least 98% sequence identity to 14-3-3zeta. In certain embodiments, the protein is 14-3-3zeta.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1F: 14-3-3z KO rats exhibit severe and early inflammatory arthritis. FIG. 1A shows the WT (n=8) and KO animals (n=8) subjected to PIA. The experiment was repeated at least 3 times. Representative body weight gain and IA score during PIA are shown. FIG. 1B shows representative pictures showing inflamed joint in WT vs. KO animals FIG. 1C shows the 3d-reconstruction of micro-CT scans of ankle and knee joints. Scale bar shows 1 mm FIG. 1D shows H&E staining of ankles harvested from WT or 14-3-3z KO rats compared for bone damage and infiltrating immune cells. Scale bar shows 200 μm. FIG. 1E shows the 14-3-3z antibody levels in synovial fluid measured in WT and KO animals using ELISA. FIGS. 1F-1G show plasma profile of IL-17A and IL-6 measured in WT and KO animals using ELISA.

FIG. 2: 14-3-3z antibodies are not responsible for the inflammatory arthritis suppression. FIG. 2A shows the 14-3-3z antibody level measured in the plasma of WT LEW rats using the in-house standardized ELISA method. FIG. 2B shows corresponding IA scores at the time of plasma collection. FIG. 2C shows the percent decrease in the plasma 14-3-3z antibodies after the incubating with GST-bound 14-3-3z beads measured using ELISA. A schematic of generating plasma and use for passive immunization is shown. FIGS. 2D-2E show the 200 μl of complete or 14-3-3z antibodies depleted plasma intravenously injected in the tail vein of WT (D, n=4) or KO (E, n=8) LEW rats. Arrows indicate the time of infusion after the pristane injection. Animals were monitored over a period of 40 days. Body weight and RA scores were measured twice a week. FIG. 2F shows fold change in 14-3-3z antibody level in sera after ld post-infusion measured using ELISA. FIG. 2G shows IA scores plotted against the sera 14-3-3z antibody levels (in the range of 100 μg/m1) of WT and KO animals infused with complete or depleted plasma.

FIGS. 3A-3H: Immunization with 14-3-3ζ in KO rats rescues the IA suppression. FIG. 3A shows experimental design of 14-3-3ζ immunization in the PIA model using 8-wk-old LEW rats. The 150 μL IFA either alone or mixed with 14-3-3ζ was injected at 1 d and 8 d postpristane (P). FIG. 3B shows the effects of 14-3-3ζ immunization on body weight and IA scores in 14-3-3ζ KO (n=5) rats compared. The experiment was repeated at least three times; a representative experiment is shown. FIG. 3C shows representative pictures of the inflamed joint in 14-3-3ζ KO rats treated with IFA or 14-3-3ζ. FIG. 3D shows the 3D reconstructions of μCT images from IFAor IFA plus 14-3-3ζ-treated animals constructed to show recovery of the trabecular bone or cortical bone thickness upon immunization. (Scale bar: 1 mm) FIG. 3E shows the H&E stain of inflamed ankles of 14-3-3ζ KO rats shows that immunization with 14-3-3ζ suppresses the infiltration of immune cells in the joints. (Scale bar: 2 mm) The inset is shown in the lower lane. (Scale bar: 200 μm.) FIGS. 3F-3H show the plasma levels of 14-3-3ζ antibody (FIG. 3F), IL-17A (FIG. 3G), and IL-6 (FIG. 3H) at the end of the study measured using ELISA. *P<0.05, ***P<0.0005, and ****P <0.0001.

FIGS. 4A-4G: Immunization with 14-3-3ζ suppresses IA in WT LEW rats. FIG. 4A shows the 8-wk-old WT LEW rats were subjected to PIA followed by injection with IFA alone or mixed with 14-protein, as shown in FIG. 3A. Animals were monitored for body weight and arthritis scores (n=4). The experiment was repeated at least three times; a representative experiment is shown. FIG. 4B shows representative pictures of inflamed joints are shown. FIGS. 4C-4D show the 14-3-3ζ antibody level in the plasma (FIG. 4C) and synovial fluid (FIG. 4D) was measured using standardized ELISA. FIG. 4E shows the plasma IL-17A level in the IFA-treated versus IFA+14-3-3ζ-treated animals measured using ELISA. FIG. 4F shows the H&E staining shows an effect of 14-3-3ζ immunization on the immune cell infiltration in the ankle joint. (Scale bar: 500 μm) The magnified image of the inset is shown on the right. (Scale bar: 200 μm.) FIG. 4G shows the 3D reconstructions of IFA- and IFA+14-3-3ζ-treated animals show the effect of 14-3-3ζ immunization on the trabecular bone and cortical bone thickness. Scale bar: 1 mm **P<0.005, and ****P<0.0001.

FIGS. 5A-5H: 14-3-3ζ promotes cortical and trabecular bone development. The μCT analysis was used to study the effect of 14-3-3ζ in gene KO and immunized animals Bones from arthritic WT and 14-3-3ζ KO rats (n=4) were analyzed to make the measurements of bone area (FIG. 5A), marrow area (FIG. 5B), tissue mineral density (FIG. 5C), cortical bone thickness (FIG. 5D), ratio of trabecular versus total bone volume (FIG. 5E), connectivity density (FIG. 5F), trabecular thickness (FIG. 5G), and trabecular separation (FIG. 5H). *P<0.05, **P<0.005, and ***P<0.0005.

FIGS. 6A-6D: 14-3-3ζ promotes collagen synthesis. FIGS. 6A-6B show Tibia (FIG. 6A) and trabecular bones (FIG. 6B) from WT and 14-3-3ζ KO—untreated, IFA−, and IFA+ 14-3-3ζ-treated arthritic animals stained for collagen with mason trichrome. (Scale bar: 500 μm and 50 μm for FIG. 6A and FIG. 6B, respectively.) FIG. 6C shows primary rat mesenchymal cells treated with different amounts of purified recombinant His-14-3-3ζ protein for 14 d, and the effect on collagen gene induction was measured using RT-qPCR. FIG. 6D shows primary rat mesenchymal cells from WT and KO cells treated with 100 ng recombinant His-14-3-3ζ for 14 d, and collagen1 gene induction was measured using RT-qPCR. *P<0.05, and ***P<0.0005.

FIGS. 7A-7G: 14-3-3ζ supplementation interferes with IL-1β signaling. FIG. 7A shows expression of proinflammatory cytokines, including Illb, Ifng, and Tnfa, measured in the circulating immune cells of WT and 14-3-3ζ KO—naïve animals using RT-qPCR. FIG. 7B shows the Illb transcript in the bone marrow of naïve 14-3-3ζ KO as compared with WT using RT-qPCR. FIG. 7C shows expression of Illb, Trap, and Opg, measured in the circulating immune cells of WT and 14-3-3ζ KO arthritic animals using RTqPCR. FIG. 7D shows the Illb transcript in the bone marrow of arthritic 14-3-3ζ KO as compared with WT animals using RT-qPCR. FIG. 7E shows expression of Illb, Illrn, and 111r2 measured in PBMC of 1 wk postimmunization in the 14-3-3ζ KO animals that received IFA alone or with 14-3-3ζ protein. FIG. 7F shows IL-1α measured in the plasma of WT and KO-naïve or arthritic animals using quantitative ELISA. FIG. 7G shows an overall model depicting the absence of 14-3-3ζ results in severe inflammation and bone damage; mainly, increased IL-1α and reduced collagen levels are observed in the naïve and IA-affected animals Immunization with 14-3-3ζ interferes with IL-1β and promotes collagen synthesis to prevent inflammation and bone damage. *P<0.05, **P<0.005, ***P<0.0005, and ****P<0.0001.

FIGS. 8A-8D: 14-3-3zeta promotes bone formation. FIG. 8A shows fluorescent labeling of bone formation in a WT mouse, and FIG. 8B shows fluorescent labeling of bone formation in a 14-3-3zeta knockout mouse. FIG. 8C shows the distance between the two layers between the two mice, and FIG. 8D shows the BFR/day between the two mice.

FIGS. 9A-9D: 14-3-3zeta regulates collagen production. FIG. 9A shows a WT mouse bone stained for collagen, and FIG. 9B shows a 14-3-3zeta^{l-} mouse bone stained for collagen. FIG. 9C shows a graph of collagen deposition comparing the WT mouse and 14-3-3zeta^{l-} mouse. FIG. 9D shows extracellular presence of 14-3-3zeta increases collagen in primary mesenchymal cells.

FIG. 10: Stimulation of 14-3-3zeta^{KO} (Ywhaz^{KO}) murine osteoblast (MC3T3) cells with Wnt3a reduces signal-induced I3-catenin nuclear translocation as compared to wildtype (Wt) cells.

FIG. 11: Stimulation of 14-3-3zeta^{KO} (Ywhaz^{KO}) MC3T3 cells with Wnt3a results in reduced alkaline phosphatase (ALP) activity as compared to wildtype (Wt) cells.

FIG. 12: Results of alizarin staining of bone sections from WT and 14-3-3zeta^{KO} (Ywhaz') animals showing reduced calcium deposits in 14-3-3zeta^{KO} (Ywhaz') animals

FIG. 13: 14-3-3zeta suppresses RANKL-stimulated signal transduction in the murine macrophage (RAW 264.7) cells. Wt and 14-3-3zeta^{KO} (Ywhaz') cells were treated with MCSF for 30 min, followed by RANKL treatment for the indicated time to study the effect on phosphorylation of ERK.

### DETAILED DESCRIPTION

Throughout this disclosure, various publications, patents, and published patent specifications are referenced by an identifying citation. The disclosures of these publications, patents, and published patent specifications are hereby incorporated by reference into the present disclosure in their entirety to more fully describe the state of the art to which this invention pertains.

In accordance with the present disclosure, it has been discovered that 14-3-3zeta promotes bone formation by increasing bone density and remodeling, and promotes osteoblasts numbers. 14-3-3zeta prevents bone damage and promotes bone growth. As described in the examples herein, deficiency of 14-3-3zeta promotes bone damage, while supplementation with 14-3-3zeta promotes bone growth. Developing a drug with an osteo-anabolic effect is highly desirable for prophylactic and therapeutic treatment for aged and immunosuppressed individuals, respectively. Thus, provided herein are various compositions and methods for promoting bone growth involving the administration of 14-3-3zeta. The present disclosure may be useful for treating or preventing bone damage, arthritis, fractures, and many other conditions.

The ubiquitously expressed 14-3-3ζ family of proteins is evolutionarily conserved from yeast to mammals 14-3-3ζ proteins are a family of conserved regulatory molecules expressed in eukaryotic cells. Their involvement in humoral and cellular immune responses has emerged through studies in drosophila and humans. The name “14-3-3” comes from the elution and migration pattern of these proteins on DEAE-cellulose chromatography and starch-gel electrophoresis. 14-3-3ζ proteins are important adaptor molecules that serve as a platform for bringing several signaling pathways closer to each other. Several roles of 14-3-3 proteins have been identified in cell cycle, cell migration, signaling, and antigenicity. There are 7 isoforms in this family in mammals All 7 isoforms (alpha/beta, delta/zeta, eta, tau, epsilon, gamma, and sigma) of 14-3-3ζ in mammals are known to bind serine or threonine phosphorylated proteins as well as several non-phosphorylated proteins. Interaction with 14-3-3ζ is known to affect stability, activity, localization of the partner protein. Both expression and function of each member of this family are under strict control due to their involvement in a multitude of signaling pathways leading cellular proliferation, differentiation, and death. Factors including posttranslational modifications, dimerization, and modifications of client proteins are also known to influence 14-3-3ζ functions.

14-3-3zeta (14-3-3ζ, also known as YWHAZ) in particular, which may also be referred to as 14-3-3ζ zeta/delta or 14-3-3ζ delta/zeta, is a protein encoded in humans by the YWHAZ gene on chromosome 8 and is a regulator of apoptotic pathways important for cell survival. 14-3-3zeta also plays an important role in a number of cancers and neurodegenerative diseases. It has previously been shown that 14-3-3zeta is an autoantigen in large vessel vasculitis, and has a role in T-cell polarization and interleukin (IL)-17A signal transduction. 14-3-3zeta has the following amino acid sequence:

(SEQ ID NO: 1)
MDKNELVQKAKLAEQAERYDDMAACMKSVTEQGAELSNEERNLLSVAYK
NVVGARRSSWRVVSSIEQKTEGAEKKQQMAREYREKIETELRDICNDVL
SLLEKFLIPNASQPESKVFYLKMKGDYYRYLAEVAAGDDKKGIVDQSQQ
AYQEAFEISKKEMQPTHPIRLGLALNFSVFYYEILNSPEKACSLAKTAF
DEAIAELDTLSEESYKDSTLIMQLLRDNLTLWTSDTQGDEAEAGEGGE
N.

As demonstrated in the examples herein, 14-3-3zeta promotes bone formation and regulates collagen production. Accordingly, administering a 14-3-3zeta protein, or a protein having at least about 95% sequence identity to 14-3-3zeta, or a 14-3-3zeta mRNA, to a subject is useful for promoting bone formation in the subject, regulating collagen production in the subject, and preventing bone damage or bone loss in the subject. It has been found that as few as two shots of a 14-3-3zeta immunization may be sufficient to provide a long-term effect in this regard. Long-term effects, measured up to 6 weeks after immunization in rats (equating to about 4 years, as 1 rat month is equivalent to 2.5 human years) have been observed. The present disclosure provides preventive as well as therapeutic benefits. For example, the 14-3-3zeta protein or mRNA may be administered to a subject having inflammatory arthritis such that one or more symptoms of the inflammatory arthritis are treated.

The 14-3-3zeta protein or mRNA can be administered through a variety of different ways, including an immunization or a skin patch. The 14-3-3zeta protein or 14-3-3zeta mRNA may be administered, for example, in the form of an injectable vaccine composition, through a transdermal patch, or through an ointment. However, as described in more detail below, many other methods of administrations are possible and encompassed within the scope of the present disclosure. The 14-3-3zeta protein or 14-3-3zeta mRNA may be particularly advantageous when administered to subjects having an elevated risk of bone fracture, such as those caused by osteoporosis, trauma, or drug-induced bone loss. The present disclosure may thus reduce the need for measures such as self-healing by immobilization in a cast, bone grafts with growth factors, and platelet rich plasma therapy.

Pharmaceutical compositions of the present disclosure comprise an effective amount of a 14-3-3zeta protein, or a protein having at least about 95% sequence identity to 14-3-3zeta, or a 14-3-3zeta mRNA, and/or additional agents, dissolved or dispersed in a pharmaceutically acceptable carrier. The phrases “pharmaceutical” or “pharmacologically acceptable” refer to molecular entities and compositions that produce no adverse, allergic, or other untoward reaction when administered to an animal, such as, for example, a human The preparation of a pharmaceutical composition that contains at least one compound or additional active ingredient will be known to those of skill in the art in light of the present disclosure, as exemplified by Remington's Pharmaceutical Sciences, 2003, incorporated herein by reference. Moreover, for animal (e.g., human) administration, it is understood that preparations should meet sterility, pyrogenicity, general safety, and purity standards as required by FDA Office of Biological Standards.

A composition disclosed herein may comprise different types of carriers depending on whether it is to be administered in solid, liquid or aerosol form, and whether it need to be sterile for such routes of administration as injection. Compositions disclosed herein can be administered intravenously, intradermally, transdermally, intrathecally, intraarterially, intraperitoneally, intranasally, intravaginally, intrarectally, intraosseously, periprosthetically, topically, intramuscularly, subcutaneously, mucosally, intraosseosly, periprosthetically, in utero, orally, topically, locally, via inhalation (e.g., aerosol inhalation), by injection, by infusion, by continuous infusion, by localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 2003, incorporated herein by reference).

The actual dosage amount of a composition disclosed herein administered to an animal or human patient can be determined by physical and physiological factors such as body weight, severity of condition, the type of disease being treated, previous or concurrent therapeutic interventions, idiopathy of the patient, and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active ingredient (i.e., the 14-3-3zeta protein or mRNA). In other embodiments, an active ingredient may comprise between about 2% to about 75% of the weight of the unit, or between about 25% to about 60%, for example, and any range derivable therein. Naturally, the amount of active ingredient(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In certain embodiments, a composition herein and/or additional agent is formulated to be administered via an alimentary route Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsules, they may be compressed into tablets, or they may be incorporated directly with the food of the diet.

In further embodiments, a composition described herein may be administered via a parenteral route. As used herein, the term “parenteral” includes routes that bypass the alimentary tract. Specifically, the pharmaceutical compositions disclosed herein may be administered, for example but not limited to, intravenously, intradermally, intramuscularly, intraarterially, intrathecally, subcutaneous, or intraperitoneally (U.S. Pat. Nos. 6,753,514, 6,613,308, 5,466,468, 5,543,158; 5,641,515; and 5,399,363 are each specifically incorporated herein by reference in their entirety).

Solutions of the compositions disclosed herein as free bases or pharmacologically acceptable salts may be prepared in water suitably mixed with a surfactant, such as hydroxypropylcellulose. Dispersions may also be prepared in glycerol, liquid polyethylene glycols and mixtures thereof, and in oils. Under ordinary conditions of storage and use, these preparations may contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In some cases, the form should be sterile and should be fluid to the extent that easy injectability exists. It should be stable under the conditions of manufacture and storage and should be preserved against the contaminating action of microorganisms, such as bacteria and fungi. The carrier can be a solvent or dispersion medium containing, for example, water, ethanol, polyol (i.e., glycerol, propylene glycol, liquid polyethylene glycol, and the like), suitable mixtures thereof, and/or vegetable oils. Proper fluidity may be maintained, for example, by the use of a coating, such as lecithin, by the maintenance of the required particle size in the case of dispersion, and/or by the use of surfactants. The prevention of the action of microorganisms can be brought about by various antibacterial and antifungal agents, such as, but not limited to, parabens, chlorobutanol, phenol, sorbic acid, thimerosal, and the like. In some cases, it may be desirable to include isotonic agents, for example, sugars or sodium chloride. Prolonged absorption of the injectable compositions can be brought about by the use in the compositions of agents delaying absorption such as, for example, aluminum monostearate or gelatin.

For parenteral administration in an aqueous solution, for example, the solution may be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed are known to those of skill in the art in light of the present disclosure. For example, one dosage may be dissolved in 1 mL of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

Sterile injectable solutions are prepared by incorporating the compositions in the required amount in the appropriate solvent with various other ingredients enumerated above, as required, followed by filtered sterilization. Generally, dispersions are prepared by incorporating the various sterilized compositions into a sterile vehicle which contains the basic dispersion medium and the required other ingredients from those enumerated above. In the case of sterile powders for the preparation of sterile injectable solutions, some methods of preparation are vacuum-drying and freeze-drying techniques which yield a powder of the active ingredient plus any additional desired ingredient from a previously sterile-filtered solution thereof. A powdered composition is combined with a liquid carrier such as, but not limited to, water or a saline solution, with or without a stabilizing agent.

In other embodiments, the compositions may be formulated for administration via various miscellaneous routes, for example, topical (i.e., transdermal) administration, mucosal administration (intranasal, vaginal, etc.), and/or via inhalation.

Pharmaceutical compositions for topical administration may include the compositions formulated for a medicated application such as an ointment, paste, cream, or powder. Ointments include all oleaginous, adsorption, emulsion, and water-soluble based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones, and luarocapram. Possible bases for compositions for topical application include polyethylene glycol, lanolin, cold cream, and petrolatum, as well as any other suitable absorption, emulsion, or water-soluble ointment base. Topical preparations may also include emulsifiers, gelling agents, and antimicrobial preservatives as necessary to preserve the composition and provide for a homogenous mixture. Transdermal administration of the compositions may also comprise the use of a patch. For example, the patch may supply one or more compositions at a predetermined rate and in a continuous manner over a fixed period of time.

In certain embodiments, the compositions may be delivered by eye drops, intranasal sprays, inhalation, and/or other aerosol delivery vehicles. Methods for delivering compositions directly to the lungs via nasal aerosol sprays has been described in U.S. Pat. Nos. 5,756,353 and 5,804,212 (each specifically incorporated herein by reference in their entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts and could be employed to deliver the compositions described herein. Likewise, transmucosal drug delivery in the form of a polytetrafluoroetheylene support matrix is described in U.S. Pat. No. 5,780,045 (specifically incorporated herein by reference in its entirety), and could be employed to deliver the compositions described herein.

It is further envisioned the compositions disclosed herein may be delivered via an aerosol. The term aerosol refers to a colloidal system of finely divided solid or liquid particles dispersed in a liquefied or pressurized gas propellant. The typical aerosol for inhalation is composed of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age and weight, as well as the severity and response of the symptoms.

In particular embodiments, the compositions described herein are useful for promoting bone growth, regulating collagen, preventing bone damage, and treating one or more symptoms of inflammatory arthritis in a subject having inflammatory arthritis. Furthermore, the compositions may be used in combination therapies. That is, the compositions may be administered concurrently with, prior to, or subsequent to one or more other desired therapeutic or medical procedures or drugs. The particular combination of therapies and procedures in the combination regimen will take into account compatibility of the therapies and/or procedures and the desired therapeutic effect to be achieved. Combination therapies include sequential, simultaneous, and separate administration of the active ingredient in a way that the therapeutic effects of the first administered procedure or drug is not entirely disappeared when the subsequent procedure or drug is administered. By way of a non-limiting example of a combination therapy, the compositions described herein can be administered in combination with one or more suitable inflammatory arthritis treatments or bone damage prevention agents such as Romosozumab, Abaloparatide, or Teraparatide.

### EXAMPLES

In these examples, the role of antigenic 14-3-3ζ was examined using animal models of IA. The results show that 14-3-3ζ is an endogenous suppressor of IA. In addition to immunological manipulations, 14-3-3ζ has a strong effect on bone remodeling. Moreover, a 14-3-3ζ-based prophylactic vaccine that reduces IA symptoms was developed. These examples show the host-protective role of antigenic 14-3-3ζ in IA suppression and show that autoantigens play an important role in inflammatory diseases.

In a model for autoimmune diseases, it was observed that 14-3-3zeta has a strong effect on bone health. Deficiency of 14-3-3zeta promotes bone loss, while supplementation promotes bone growth in the animal model. Advantageously, the data shows that two shots of 14-3-3zeta immunization resulted in a long-term effect, at least until the end of the experiment (>45 days).

In these examples, it is demonstrated that 14-3-3ζ knockout (KO) rats develop early-onset severe arthritis in two independent models of IA, pristane-induced arthritis and collagen-induced arthritis. Arthritic 14-3-3ζ KO animals showed an increase in bone loss and immune cell infiltration in synovial joints. Induction of arthritis coincided with the loss of anti-14-3-3ζ antibodies; however, rescue experiments to supplement the 14-3-3ζ antibody by passive immunization did not suppress arthritis. Instead, 14-3-3ζ immunization during the presymptomatic phase resulted in significant suppression of arthritis in both wild-type and 14-3-3ζ KO animals Mechanistically, 14-3-3ζ KO rats exhibited elevated inflammatory gene signatures at the messenger RNA and protein levels, particularly for IL-1β. Furthermore, the immunization with recombinant 14-3-3ζ protein suppressed IL-1α levels, significantly increased anti-14-3-3ζ antibody levels and collagen production, and preserved bone quality. The 14-3-3ζ protein increased collagen expression in primary rat mesenchymal cells. Together, these findings indicate that 14-3-3ζ causes immune suppression and extracellular remodeling, which lead to a previously unrecognized IA-suppressive function.

Rheumatoid arthritis (RA) is a chronic autoimmune disease associated with increased innate and adaptive immune responses. Antigens activate T and B cells, leading to increased production of cytokines and antibodies, a characteristic of seropositive RA. Increased rheumatoid factor and anticitrullinated antibodies correlate with RA disease activity. However, the loss of protective autoantibodies or autoantigens in immune diseases remains unclear. It is believed that autoantibodies can deplete antigens or activate complement pathways to produce immune suppression. Therefore, the disruption of homeostasis between natural antibodies and target antigen expression generates a bias in favor of pathogenic pathways responsible for autoimmune diseases. In the absence of antigen-based protective immunity, autoantigens can promote pathogenesis, either directly or by neutralizing protective mechanisms. Most autoantigens in RA are cytosolic proteins exposed to the external environment due to cell death or NETosis. Antigen-stimulated innate and adaptive immune responses often support inflammation. However, antigen-specific immunotherapy is a useful strategy in treating autoimmune diseases, and several clinical trials are testing for induction of antigen-specific tolerance in RA. It is important to understand the role of protective mechanisms responsible for RA suppression.

The 14-3-3ζ protein is an adaptor that regulates cellular signaling by binding to a wide range of proteins. Changes in 14-3-3ζ expression levels are associated with cancer and neurological and cardiovascular pathologies. The 14-3-3ζ genetic variants exhibit RASOpathies, particularly the cardiofaciocutaneous syndrome. 14-3-3ζ regulates immune responses via antigen presentation and extracellular signaling. 14-3-3ζ is an antigen in thoracic aortic aneurysms associated with large vessel vasculitis, and promotes human T-cell polarization in favor of T helper (Th)1 and Th17 cells. In accordance with the present disclosure, 14-3-3ζ has an immunogenic function with pathologic autoimmune consequences. In these examples, the role of 14-3-3ζ and its antigenic function in animal models of inflammatory arthritis was investated.

The effect of 14-3-3ζ on IA on 8-wk-old arthritissusceptible Lewis (LEW) rats that were intradermally injected with either pristane or type-II collagen to induce pristaneinduced arthritis (PIA) or collagen-induced arthritis (CIA), respectively, was evaluated. Wild-type (WT) and global 14-3-3ζ knockout (KO) LEW rats were used to examine the function of endogenous 14-3-3ζ. Furthermore, animals were immunized with incomplete Freund's adjuvant (IFA) mixed with purified 14-3-3ζ to study its immunogenic role in IA progression. Contrary to a hypothesis, 14-3-3ζ KO rats developed severe joint inflammation and lost a significant amount of bone and body weight when tested in PIA and CIA models. Importantly, it was observed that 14-3-3ζ immunization reduced joint inflammation while preserving bone and body weight. A negative trend was found between circulating 14-3-3ζ antibody levels and inflammatory arthritis scores. However, replenishing the antibody by passive immunization was ineffective in suppressing the inflammation, indicating that suppression of arthritis required an active immunogenic function of 14-3-3ζ. The long-term effects of 14-3-3ζ immunization included suppressing proinflammatory cytokines and promoting collagen synthesis and bone preservation. Mainly, it was observed that 14-3-3ζ downregulates interleukin (IL)-1β and up-regulates the IL-1 receptor antagonist, thereby causing arthritis suppression. The results show that 14-3-3ζ is a suppressor of inflammatory arthritis, which has therapeutic implications in RA.

### Results

### 14-3-3ζ KO Rats Develop Early and Severe RA

To investigate the role of endogenous 14-3-3ζ protein in the pathogenesis of IA, 14-3-3ζ global KO rats were generated using a CRISPR-Cas9 technology. A 58-bp deletion in exon 3 of the 14-3-3ζ gene resulted in global KO in LEW rats. The KO rats are not good breeders. Therefore, heterozygous animals were used for breeding. Loss of 14-3-3ζ resulted in reduced anti-14-3-3ζ antibody levels in the KO animals Antibody levels were measured at various dilutions of plasma using in-house standardized enzyme linked immunosorbent assay (ELISA), and a ˜90% loss of 14-3-3ζ antibody was observed in KO animals compared with WT. The remaining 10% antibody reflected the nonspecific binding, likely due to a high degree of sequence conservation in 14-3-3ζ family members. To account for 14-3-3ζ specificity in antibody measurements, all future ELISA values were subtracted by 10%. The absence of 14-3-3ζ in the KO animals did not affect 14-3-3η antibodies in the plasma.

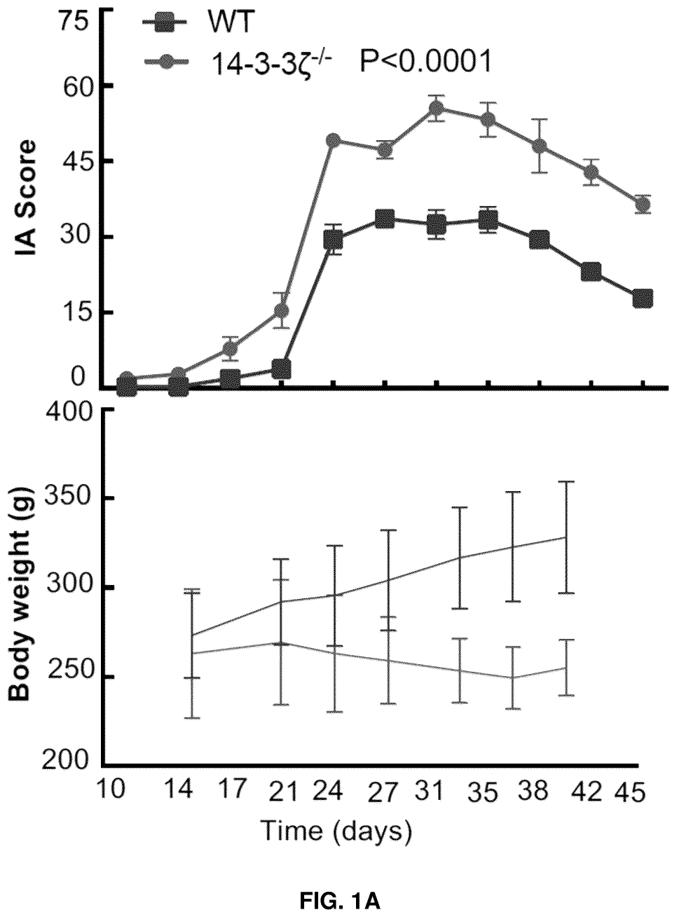

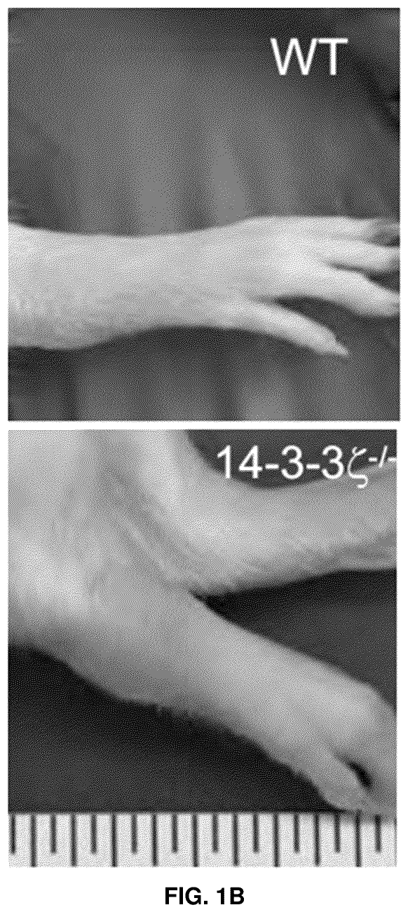

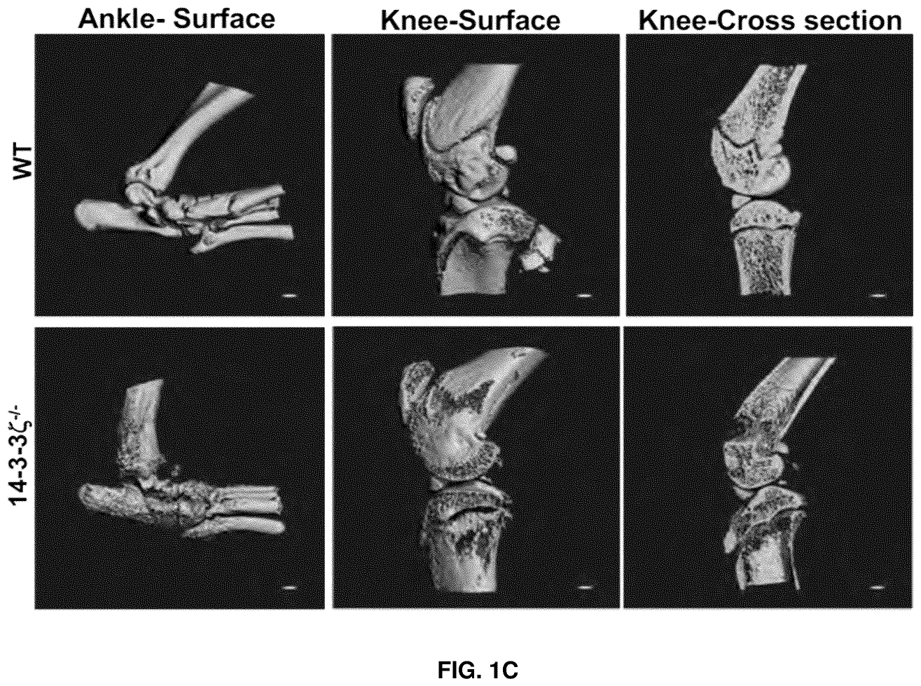

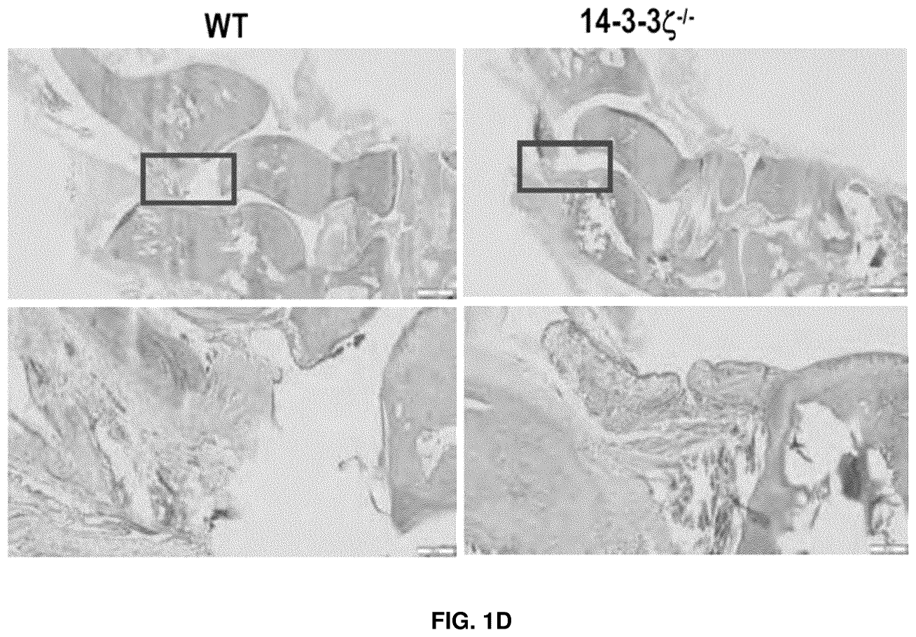

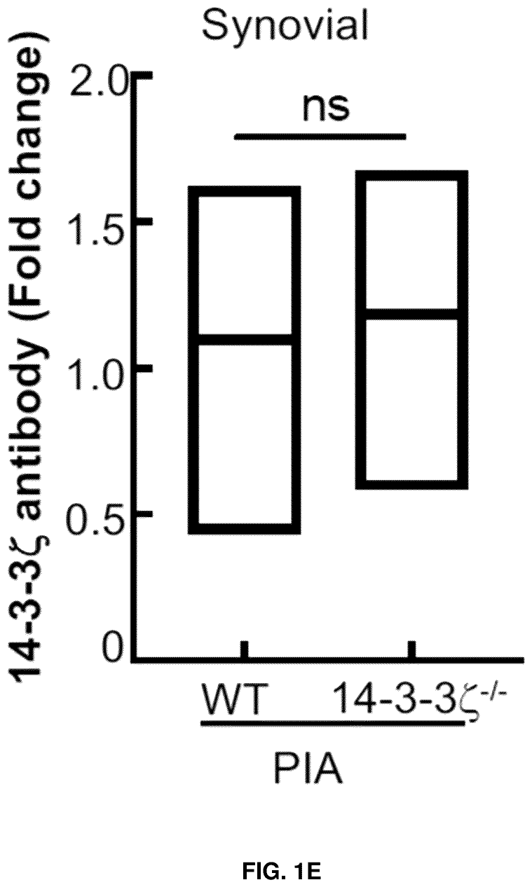

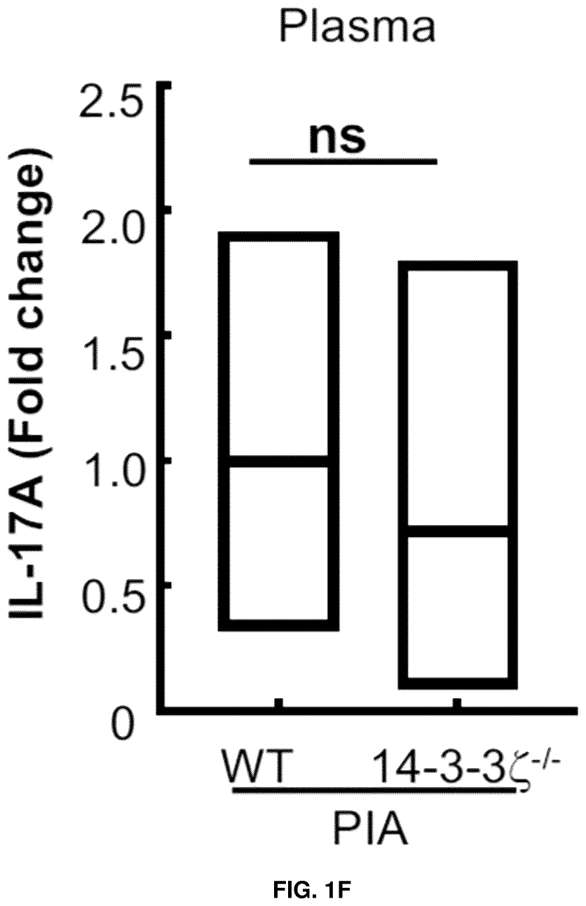

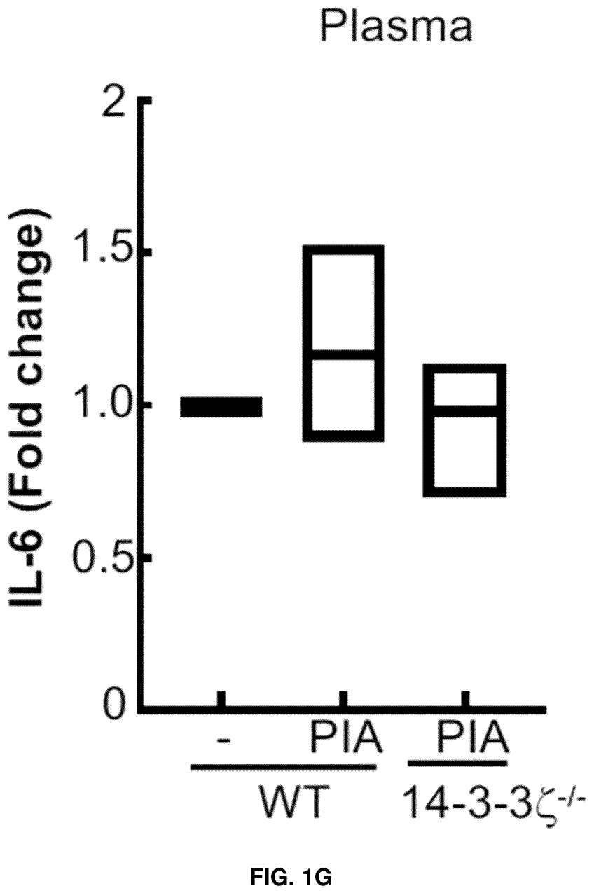

Animal models of IA share several features with human RA, including T-cell dominance. The initiation of arthritis in LEW rats resulted in a typical three-phase disease, including an asymptomatic period and severe joint inflammation, followed by a resolution phase in which inflammation starts to subside. A 0 to 80 scoring system of arthritis was used in these examples. Compared with WT, pristane induced an early-onset joint inflammation with significantly higher arthritic scores in 14-3-3ζ KO animals The increase in arthritis scores correlated with the decreased weight gain during the experiment (FIG. 1A). Compared with WT, the 14-3-3ζ KO animals showed highly inflamed swollen joints, which accounted for higher arthritis scores (FIG. 1B). The arthritic ankle and knee joints were studied using micro computed tomography (μCT). The μCT analysis of ankle and knee joints showed that the bones from 14-3-3ζ KO animals had increased abnormal ectopic bone and significant bone surface damage. The increased bone erosion associated with growth plate and articular surfaces was strikingly prominent in the 14-3-3ζ KO animals The proximal tibia and femur showed a significant trabecular bone loss in 14-3-3ζ KO rats (FIG. 1C).

Histological analyses of ankle and knee joints showed an increased immune cell infiltration in the synovium of 14-3-3ζ KO animals (FIG. 1D). There was no significant difference in the 14-3-3ζ antibody levels in the synovial fluid of KO animals compared with WT animals (FIG. 1E). The serological cytokine analysis showed no substantial changes in IL-17A or IL-6 in 14-3-3ζ KO animals (FIG. 1F and FIG. 1G). To determine if arthritis susceptibility was PIA specific, 14-3-3ζ KO was examined in the CIA model. Like PIA, 14-3-3ζ KO animals developed much higher inflammation of joints resulting in significant arthritis scores. These results indicate that endogenous 14-3-3ζ has a unique function in the suppression of IA.

### 14-3-3C Antibodies are not Responsible for Arthritis Suppression

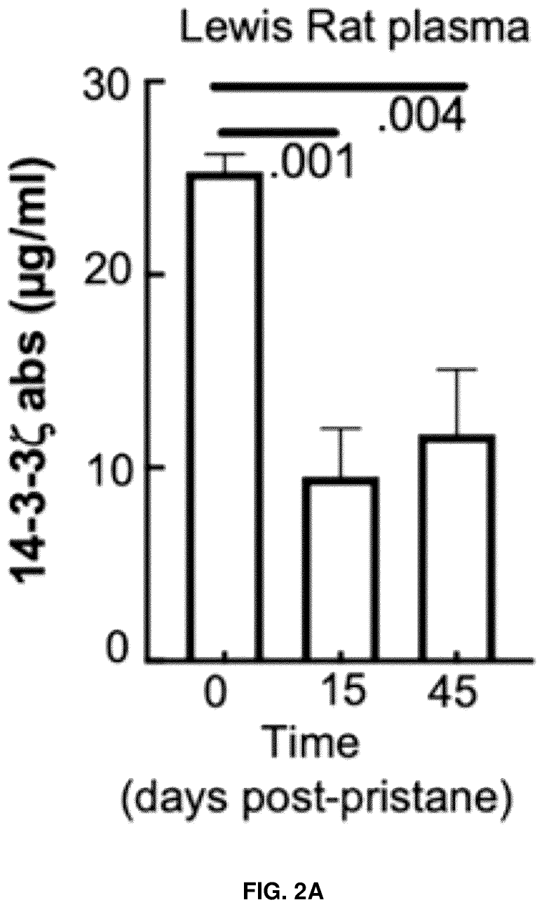

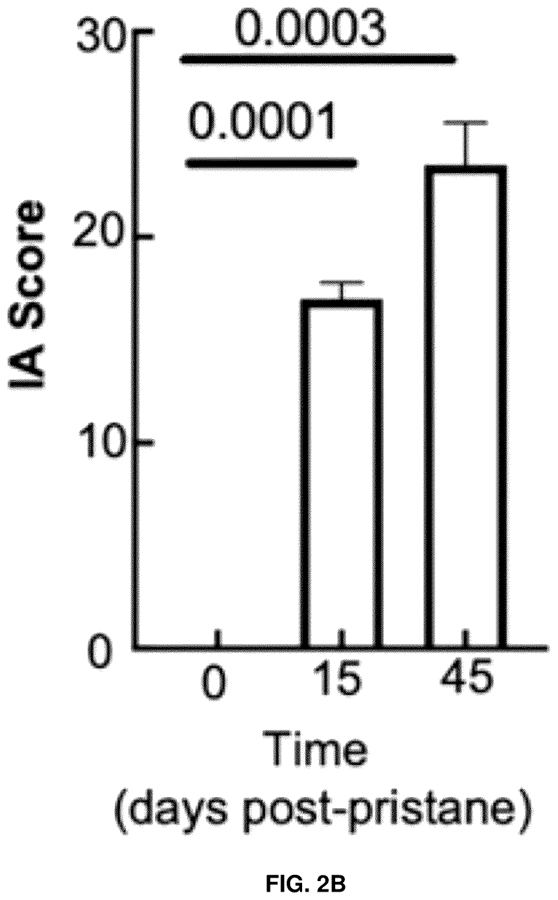

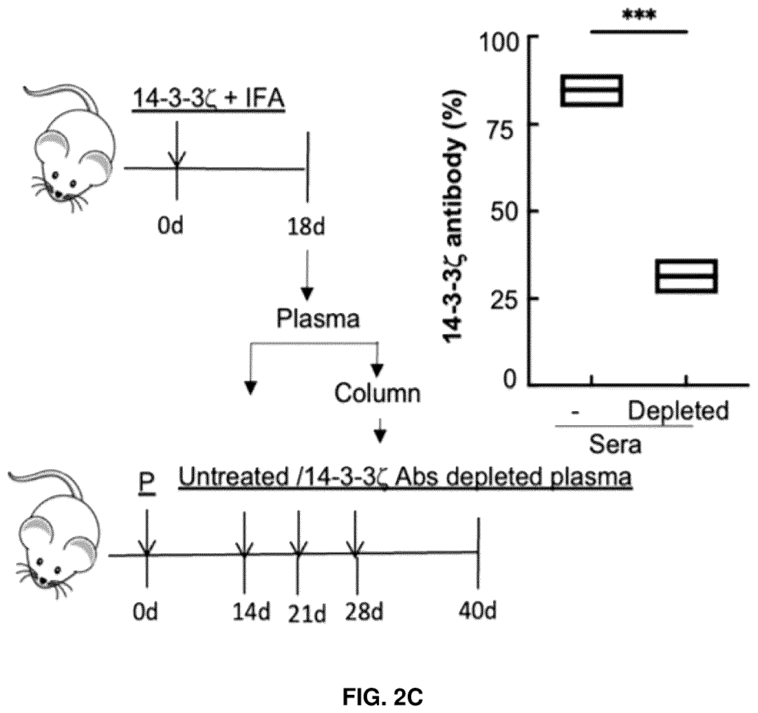

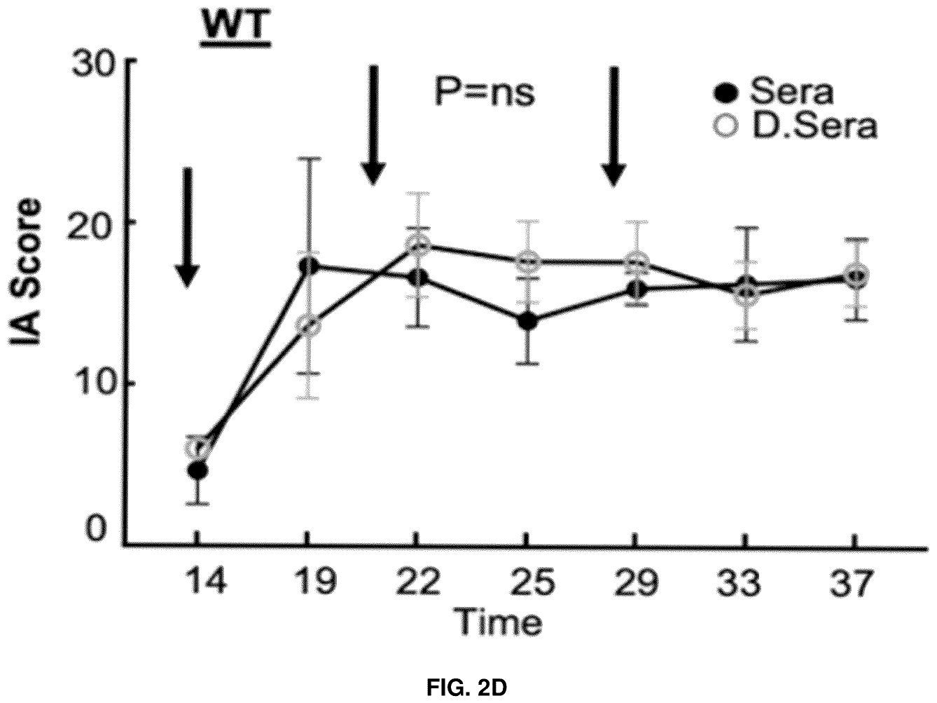

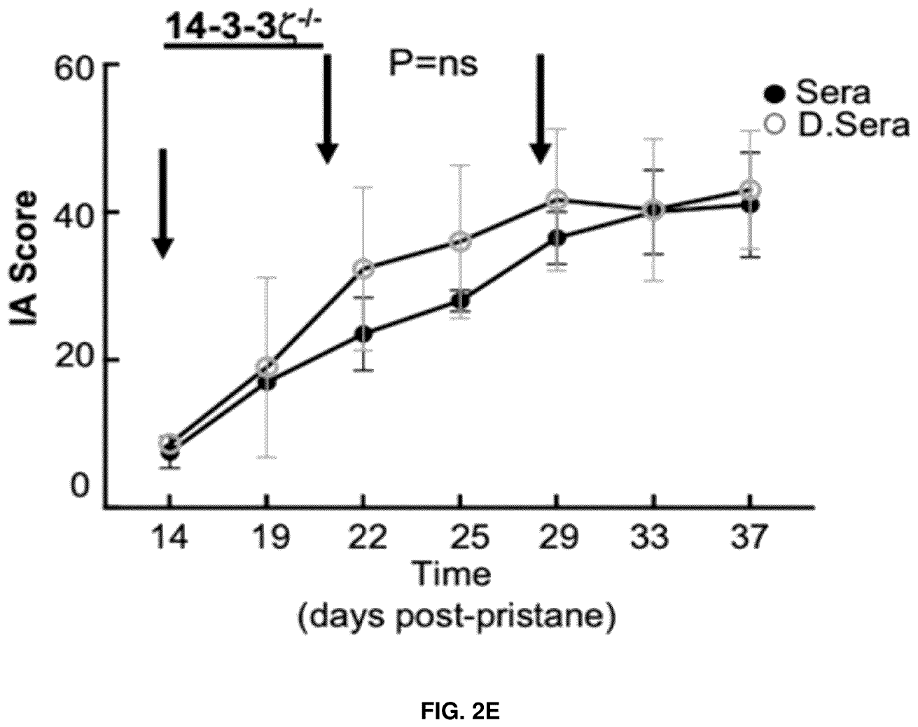

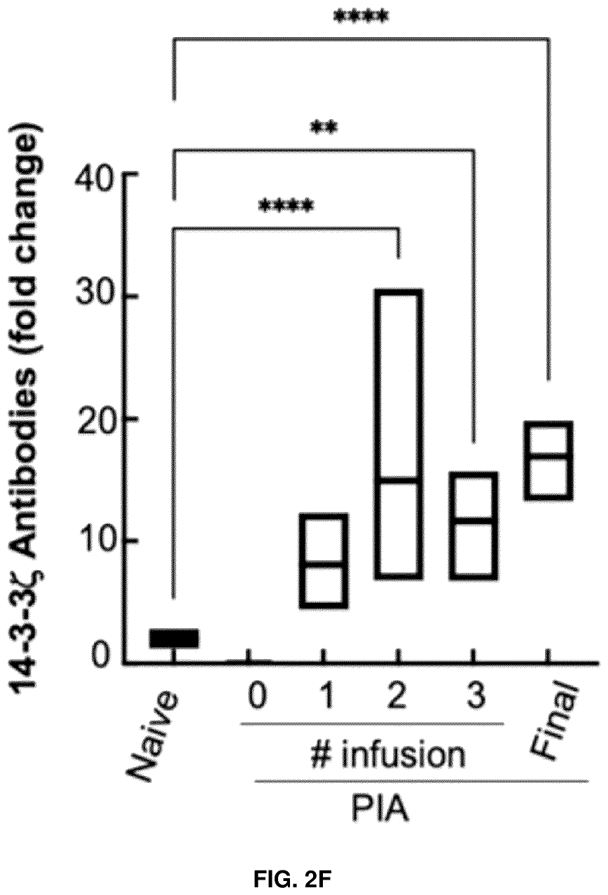

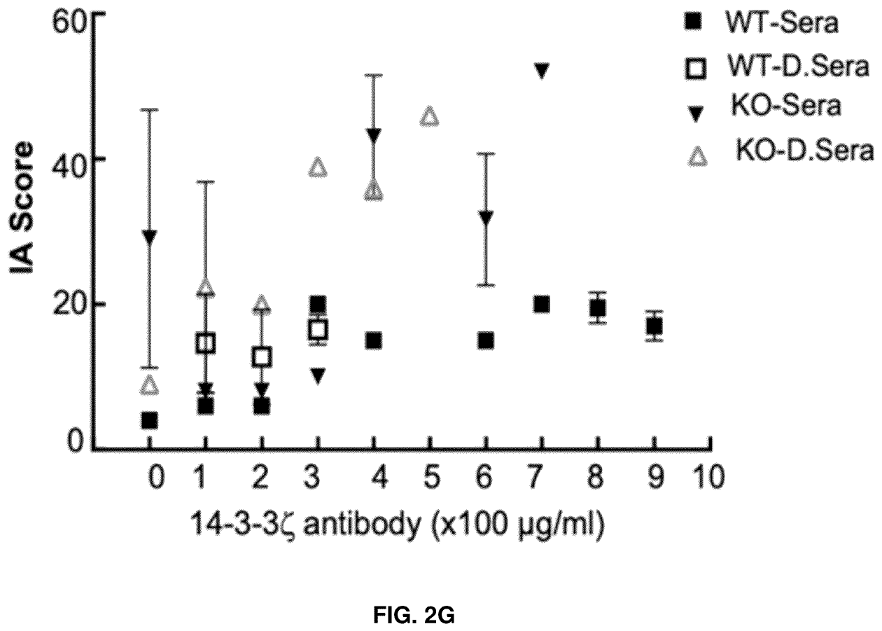

Higher arthritis scores and reduced 14-3-3ζ antibodies in the KO animals raised a question as to whether a decrease in 14-3-3ζ antibodies is responsible for severe arthritis. How the progression of arthritis impacted the levels of 14-3-3ζ antibodies in WT LEW rats was examined 14-3-3ζ antibodies were measured in the plasma of WT animals injected with pristine at the onset of symptoms (15 d) and resolution (˜45 d). The level of the 14-3-3ζ antibody significantly decreased by the time experimental arthritis symptoms were visible and remained low for the duration of the study (FIGS. 2A-2B). Arthritis scores and 14-3-3ζ antibody levels were inversely correlated, and their relationship was statistically significant. Accordingly, a protective role of 14-3-3ζ antibodies in IA was investigated.

To test the role of the 14-3-3ζ antibody, a passive antibody administration was performed by convalescent plasma transfusion to WT and KO animals after arthritis initiation with pristane. antibody-containing plasma was obtained from 18-d postimmunized WT LEW rats. Half of the pooled plasma was processed to remove 14-3-3ζ antibody by incubating with a column bound purified 14-3-3ζ protein, which resulted in >75% reduction in the antibodies (FIG. 2C). Original plasma or one with depleted 14-3-3ζ antibodies was used for the infusion during arthritis progression, per the scheme shown in FIG. 2C. At 14, 21, and 28 d post-pristane, three intravenous injections of plasma were performed (FIG. 2C) Animals were regularly examined for arthritis and body weight. There were no immediate or long-term effects of passive plasma transfer on arthritis progression, as shown in the arthritis scores of WT or 14-3-3ζ KO animals (FIGS. 2D, 2E). The 14-3-3ζ antibody levels measured 1 d postinfusion showed restoration of its levels (FIG. 2F). Furthermore, regardless of the type of plasma infused, no correlation between serum antibody levels and IA score was observed in WT or KO animals (FIG. 2G). Additionally, infusion at earlier time points did not result in arthritis suppression. These results indicate that 14-3-3ζ antibodies do not contribute to the suppression of arthritis.

Immunization with 14-3-3ζ prevents IA progression in 14-3-3ζ KO rats

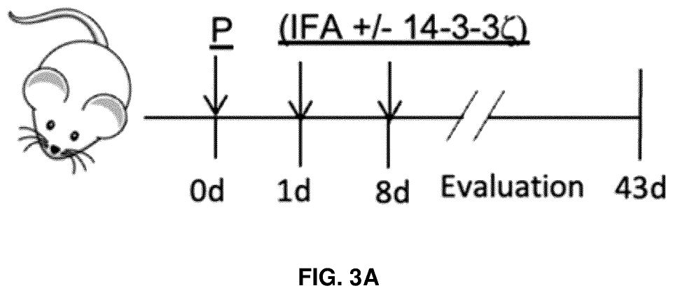

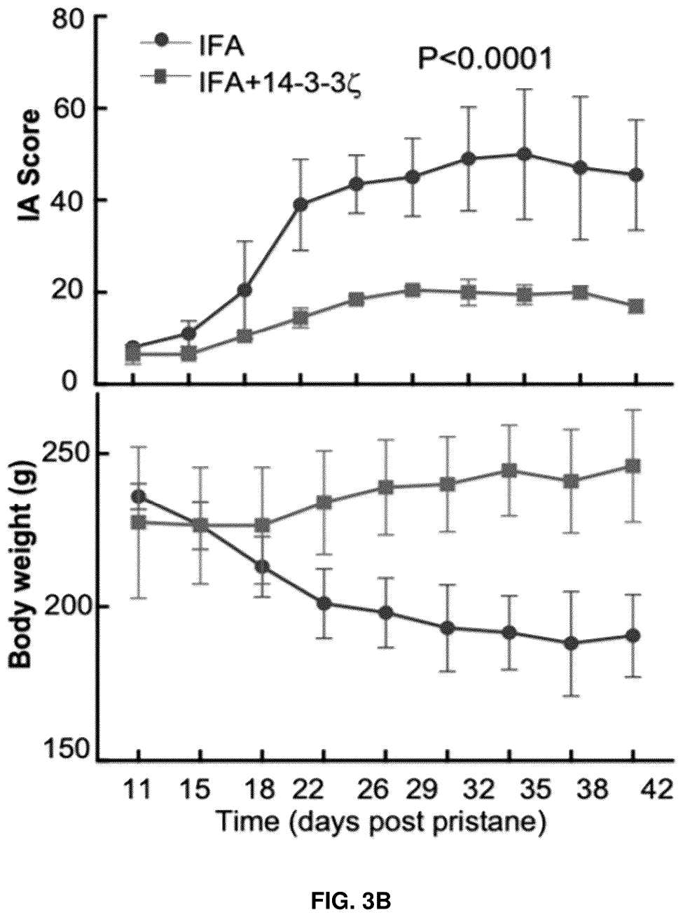

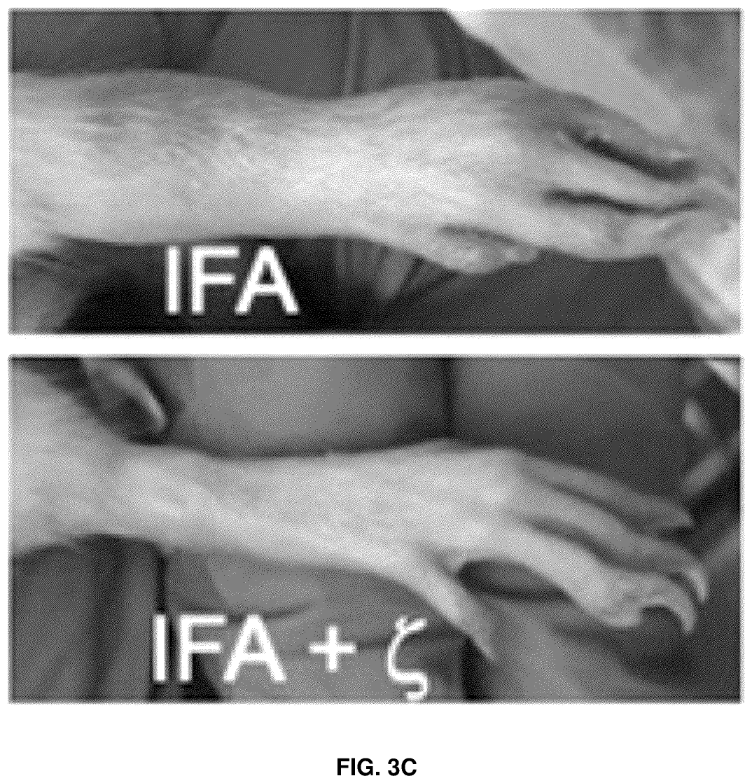

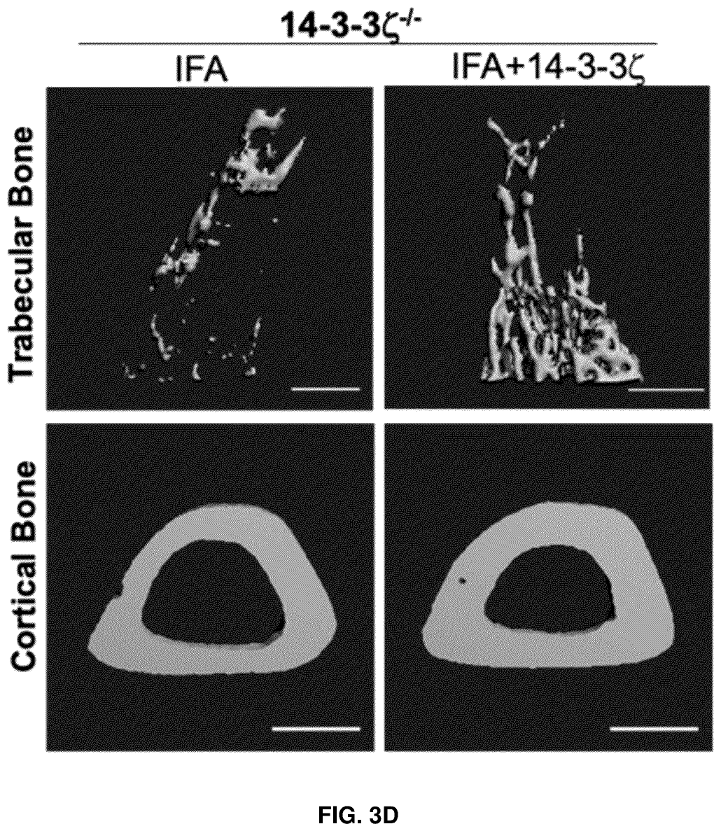

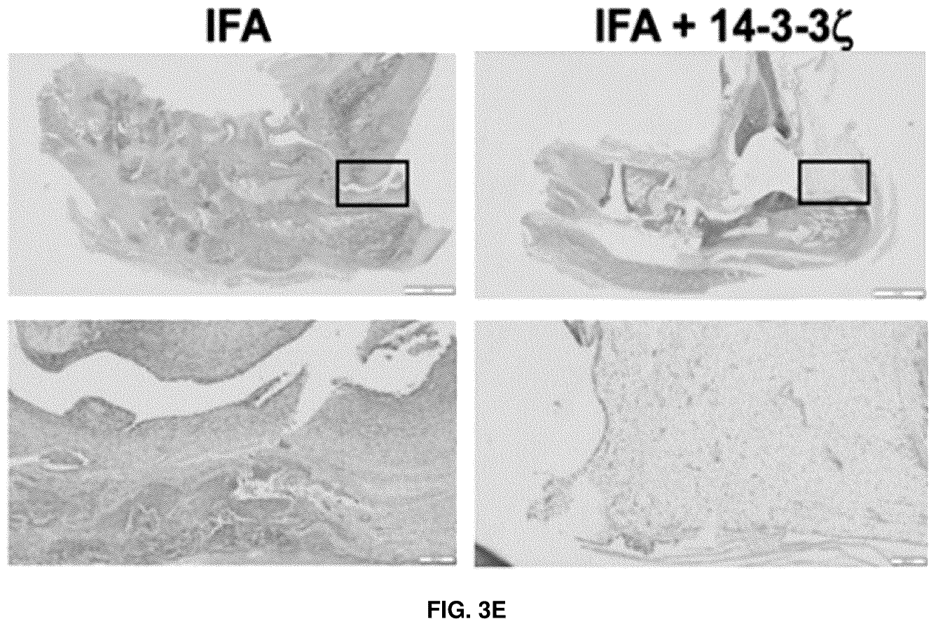

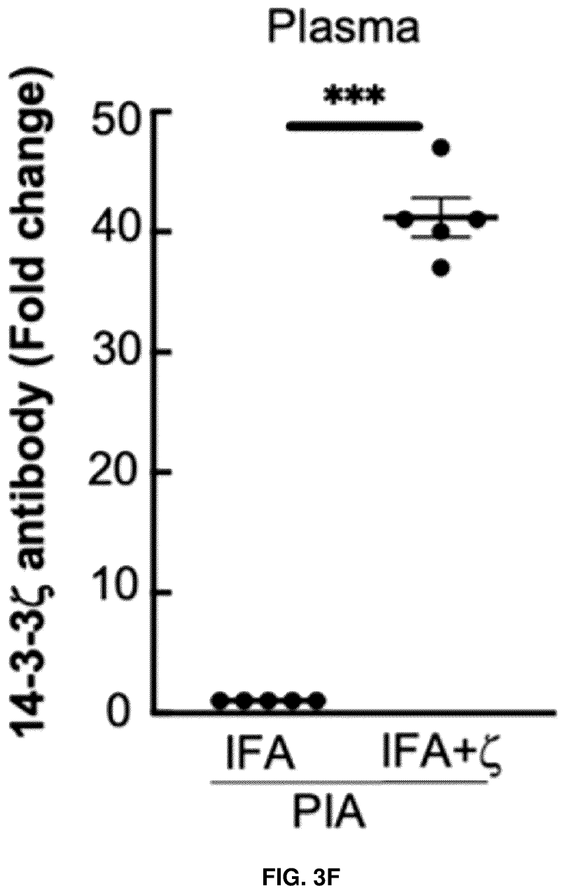

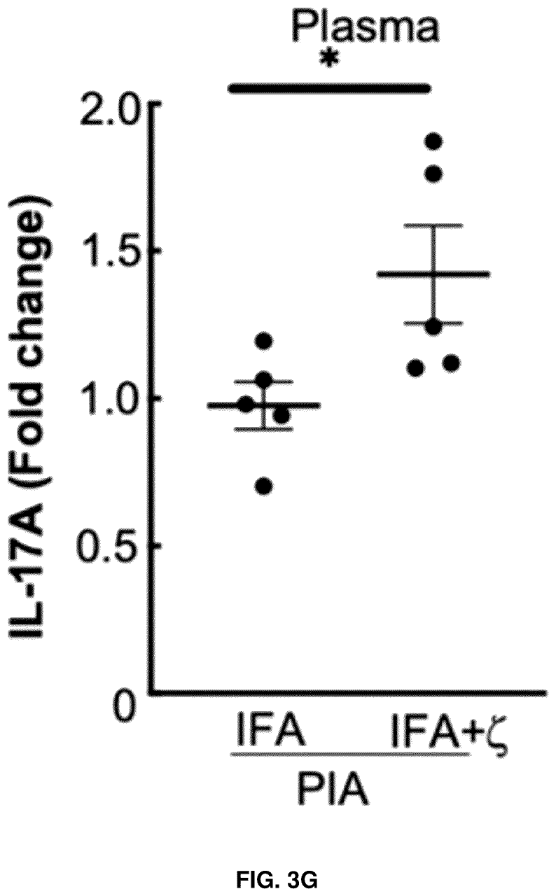

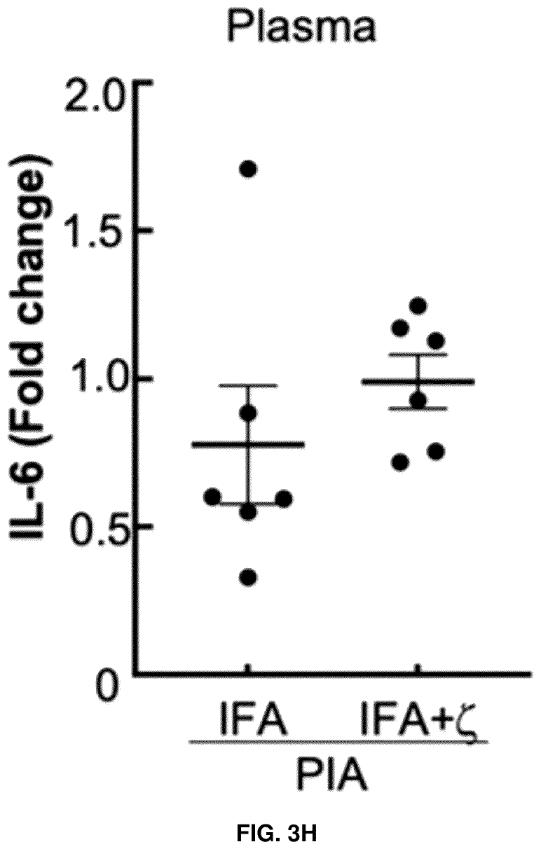

After ruling out the protective function of antibodies, whether 14-3-3ζ's antigenic role is responsible for arthritis suppression was evaluated. 14-3-3ζ immunization was performed in the KO animals, since they exhibit severe arthritis in both PIA and CIA models of IA. Animals were divided into two groups; one received purified human 14-3-3ζ mixed with IFA, while the other received only IFA (FIG. 3A). A two-dose immunization strategy—the first at 1 d and booster at 8 d postpristane—was adopted, and the effect on the arthritis progression was measured. Compared with IFA alone, immunization with 14-3-3ζ significantly reduced the disease progression, as evidenced by lower arthritic scores in KO animals Decreased arthritis severity in 14-3-3ζ-treated animals correlated with the increase in weight gain during the study (FIG. 3B). Limb swelling was visibly reduced in the animals that received 14-3-3ζ (FIG. 3C). The μtCT analysis of the tibia bones from 14-3-3ζ KO animals showed a significant loss of trabecular bone and cortical bone thickness in the IFA-treated animals Importantly, 14-3-3ζ immunization protected both the trabecular bone and cortical bone (FIG. 3D).

The histological assessment of the inflamed joints from IFA-treated animals showed a significant infiltration of immune cells. In comparison, 14-3-3ζ-treated animals had reduced immune cells in the joints (FIG. 3E), further demonstrating the immune suppression. To verify this observation, the effect of 14-3-3ζ immunization was evaluated in the CIA model. Like PIA, 14-3-3ζ KO in the CIA model showed severely inflamed joints and high arthritis scores, which were significantly reduced in animals immunized with 14-3-3ζ.

To examine the immunogenic function, how 14-3-3ζ treatment affects cytokine and antibody induction in vivo was measured. The 14-3-3ζ immunization resulted in a significant increase of plasma antibody and IL-17A but not in IL-6 (FIGS. 3F-3H). Similar to plasma, 14-3-3ζ antibody levels in the synovial fluid of the 14-3-3ζ-immunized animals were also increased. In comparison with >300-fold induction in the 14-3-3ζ antibody in WT, the effect on 14-3-3i antibody was marginal. To examine side effects of 14-3-3ζ immunogenicity, LEW rats were subjected to two doses of immunization with IFA alone or with 14-3-3ζ. In the absence of pristane, 14-3-3ζ immunization or IFA alone had minimal effect on joint inflammation or any other physical symptoms. Since IL-17A increase is associated with RA, whether a 14-3-3ζ-stimulated IL-17A increase is sufficient to cause arthritis was investigated. Wistar rats were subjected to pristane and the immunization strategy, as shown in FIG. 3A. Like LEW rats, 14-3-3ζ immunization of Wistar rats resulted in the significant antibody and IL-17A induction but no significant change in the arthritic score. Overall, these results show that in vivo antigenic function of 14-3-3ζ prevents inflammatory arthritis in the LEW rat model of PIA and CIA.

### 14-3-3ζ Immunization Suppresses Arthritis in WT LEW Rats

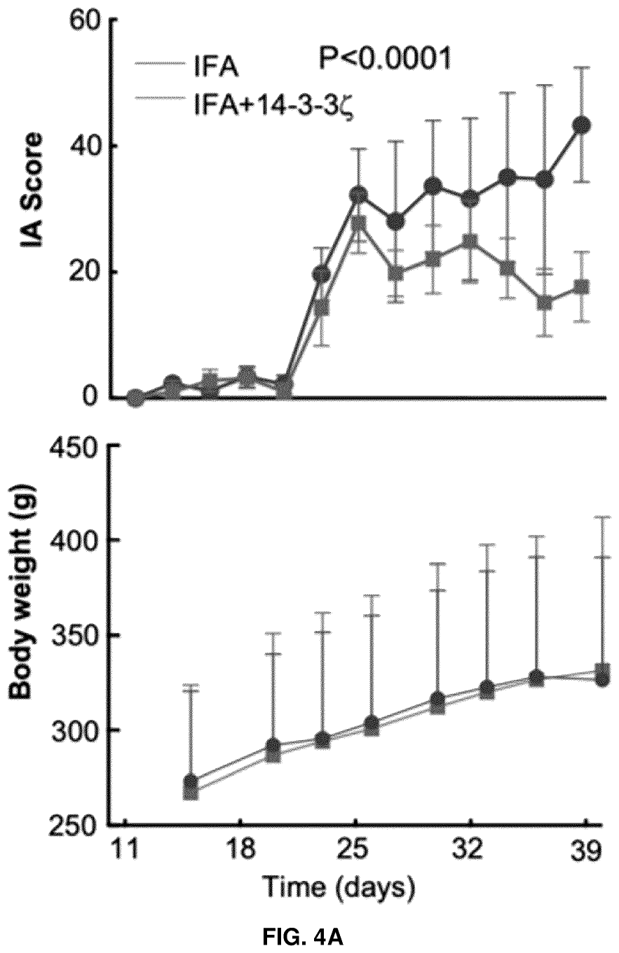

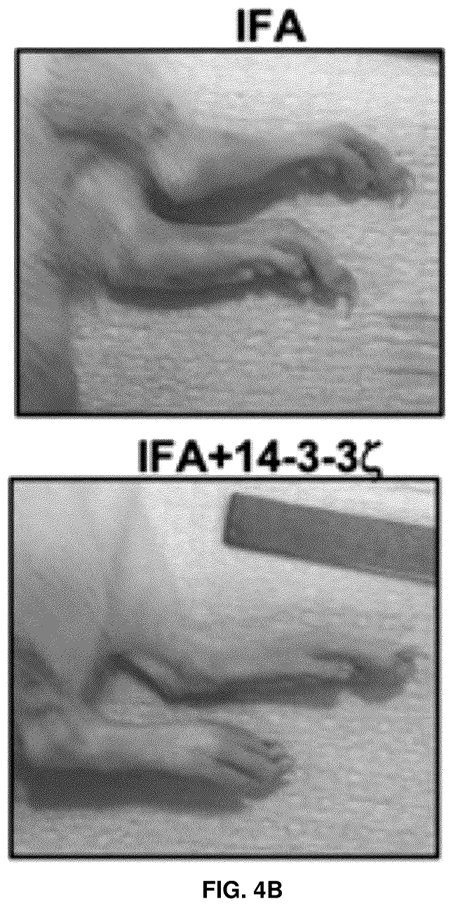

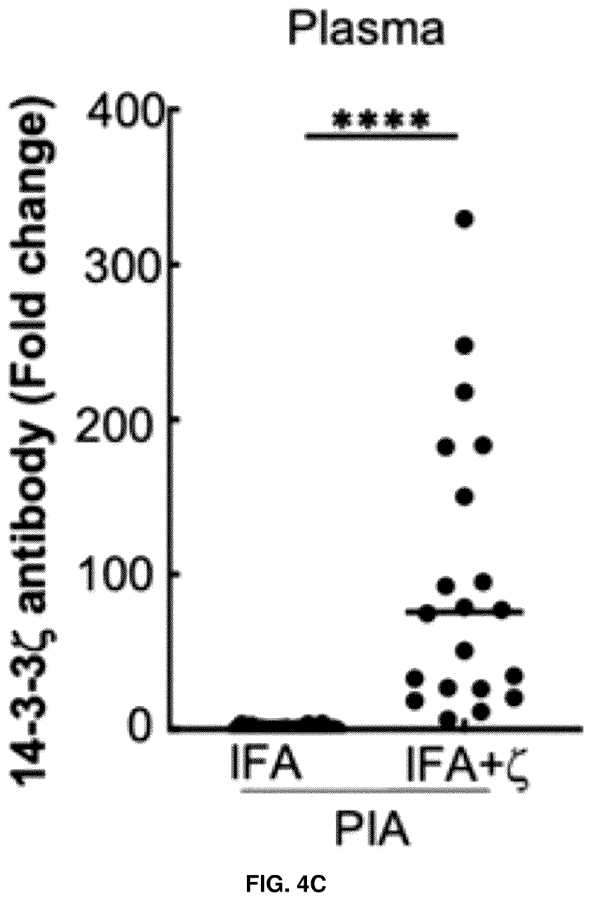

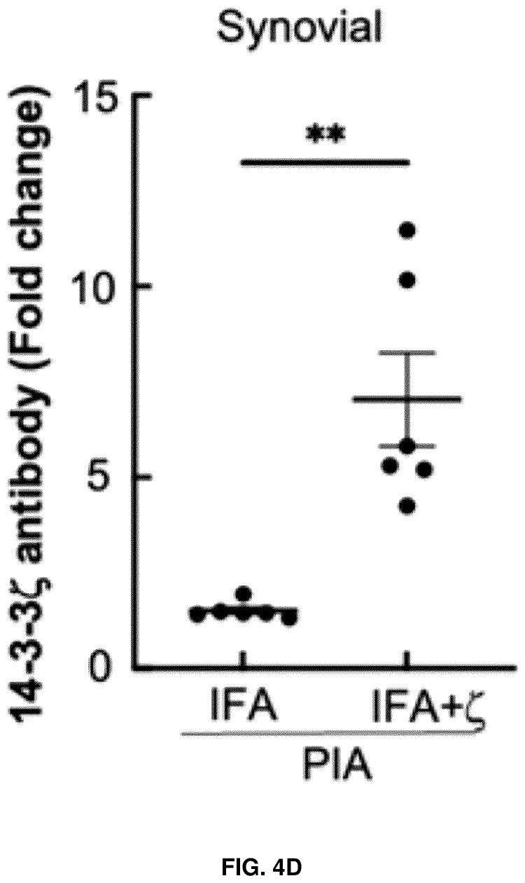

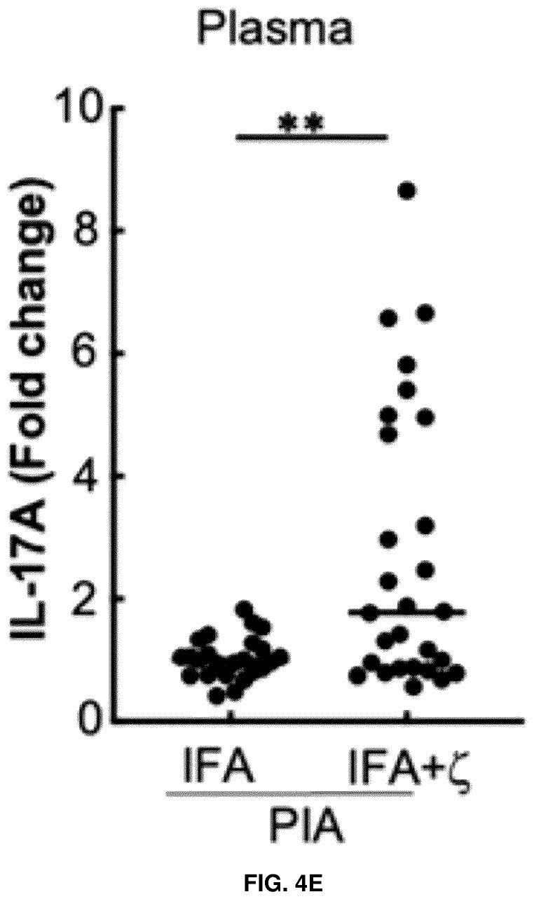

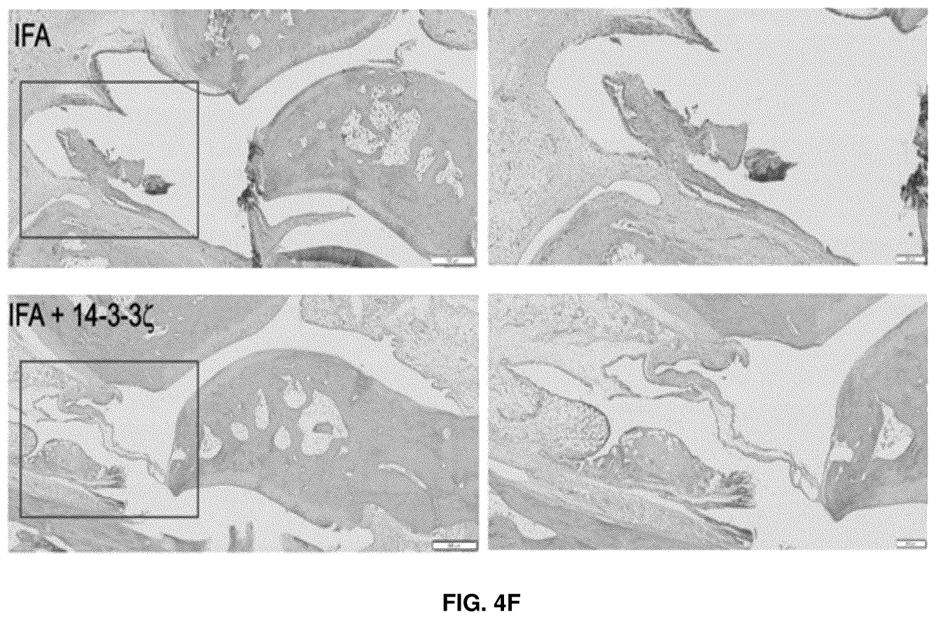

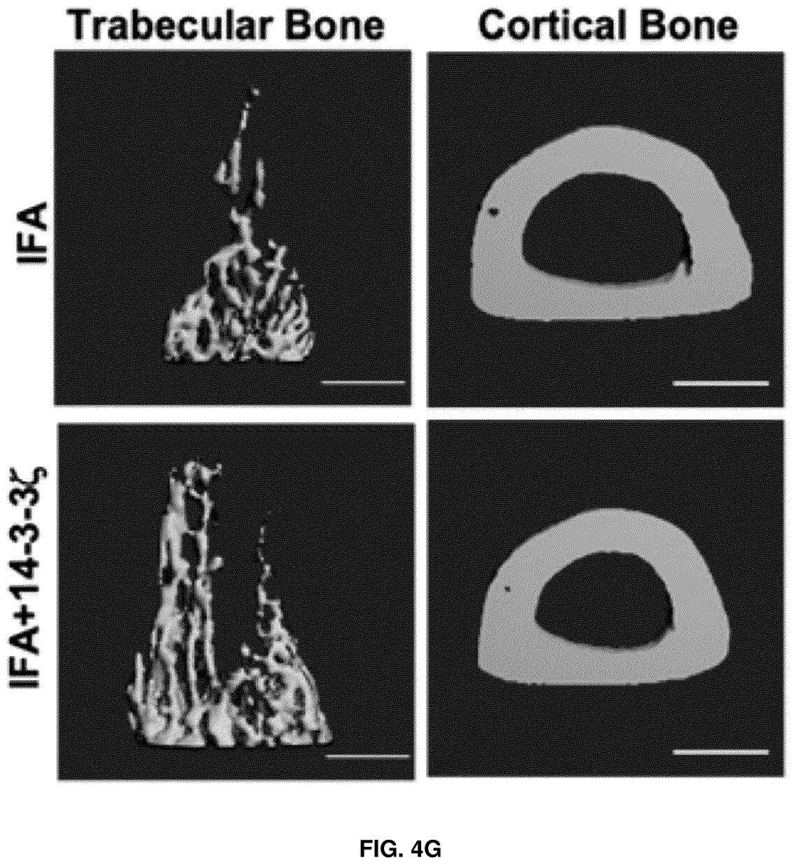

WT LEW animals were used to examine the effect of 14-3-3ζ-mediated IA suppression on mild arthritic disease. The IFA-treated WT LEW rats showed a significant increase in joint inflammation by 28 d postpristane injection, which remained high until the end of the experiment at 45 d. Comparatively, the animals immunized with IFA plus 14-3-3ζ showed a substantial reduction in arthritis scores. No significant difference in body weight was observed between the two groups (FIG. 4A). Visually, 14-3-3ζ- immunized animals had less swollen joints (FIG. 4B). Like KO, 14-3-3ζ immunization induced robust antibody production as measured in the plasma and synovial fluid of the arthritic WT animals (FIGS. 4C, 4D). A significant increase in the IL-17A level was observed in 14-3-3ζ-immunized rats (FIG. 4E).

Histological analyses of affected ankles showed significant damage to the synovium, and inflammatory cell infiltrates in the joints of IFA-treated rats, compared with animals that were immunized with 14-3-3ζ (FIG. 4F). The μCT analysis of the tibia also confirmed improvement in the trabecular bone and cortical bone thickness upon 14-3-3ζ immunization (FIG. 4G). These results confirmed that 14-3-3ζ immunization reduces immune cell infiltration in the synovium and improves arthritis scores in rats.

### 14-3-3ζ Prevents Bone Damage

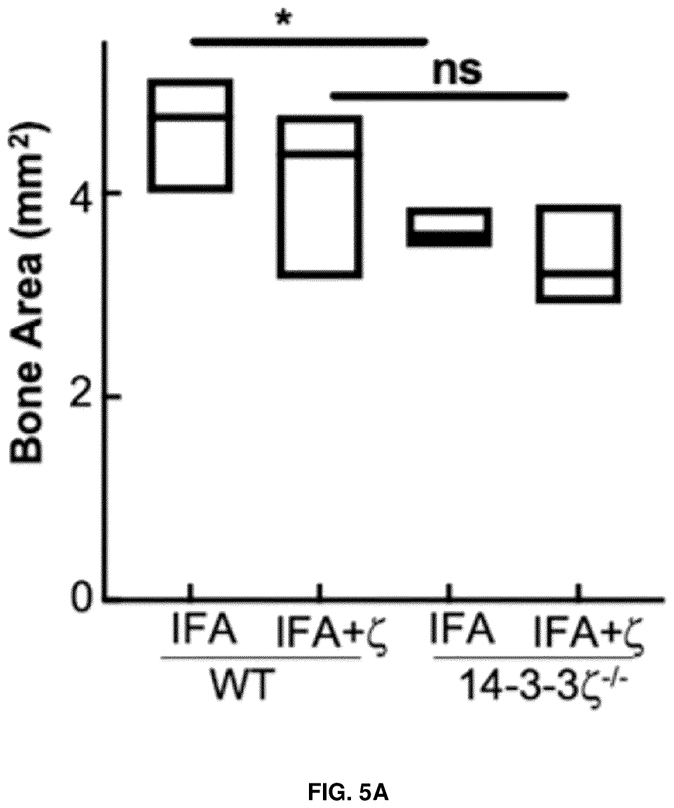

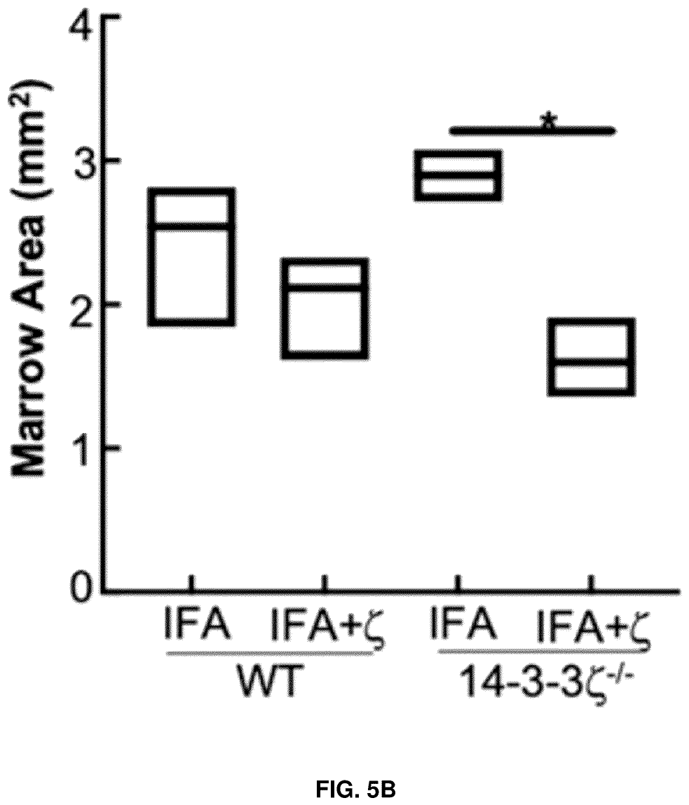

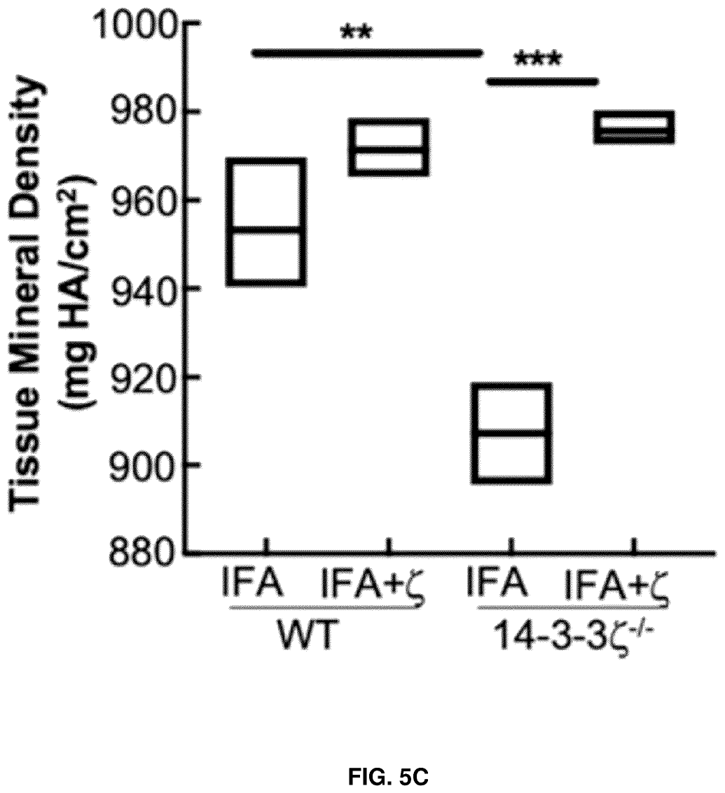

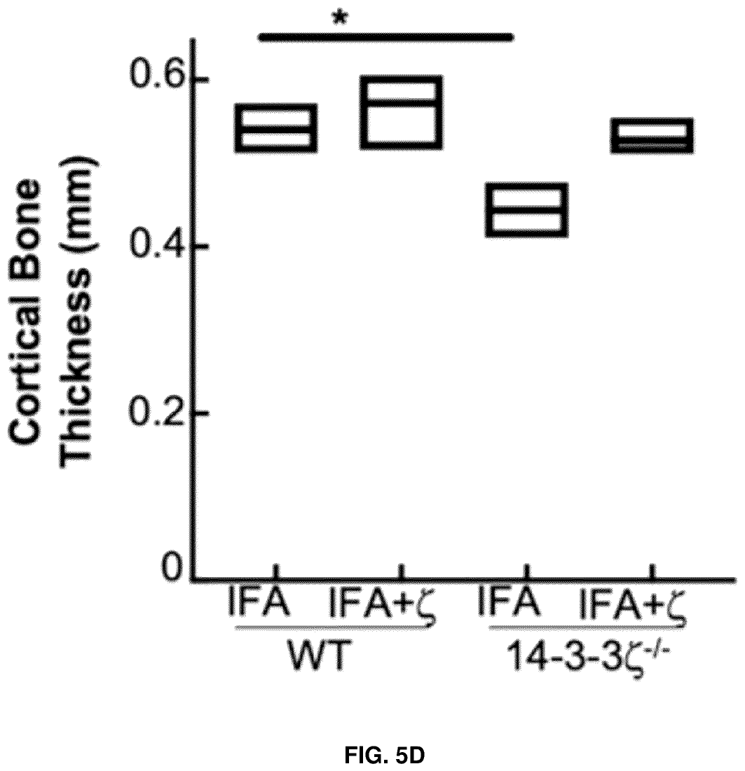

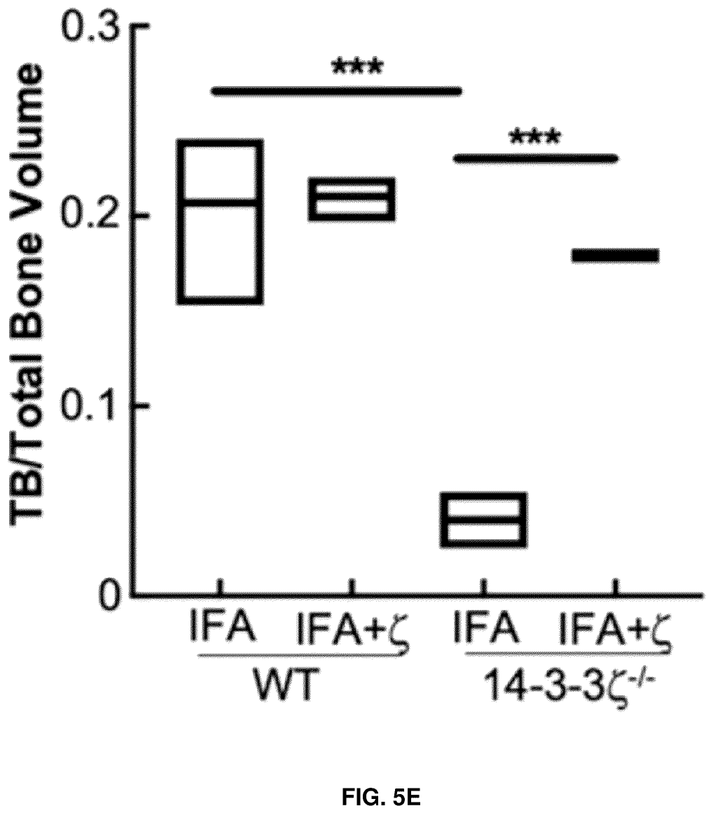

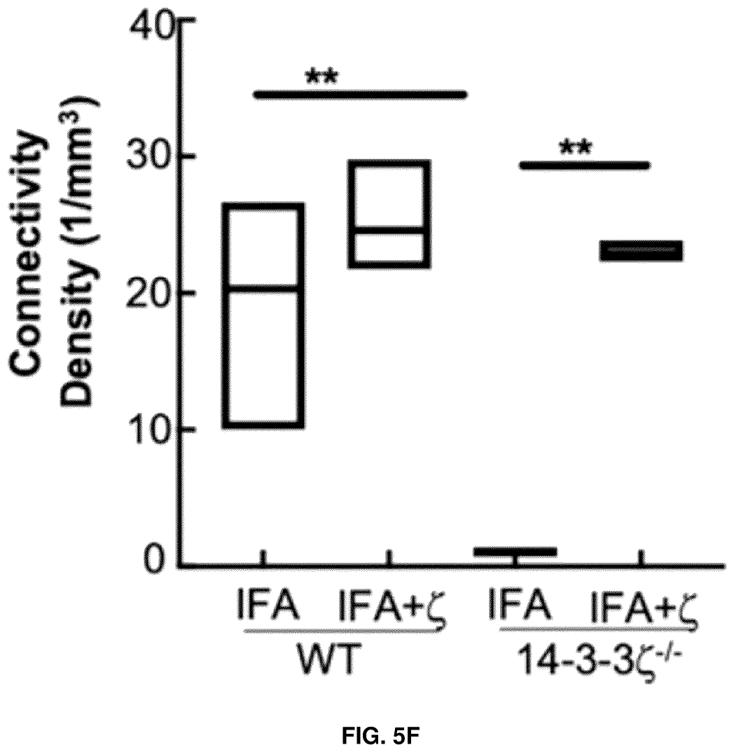

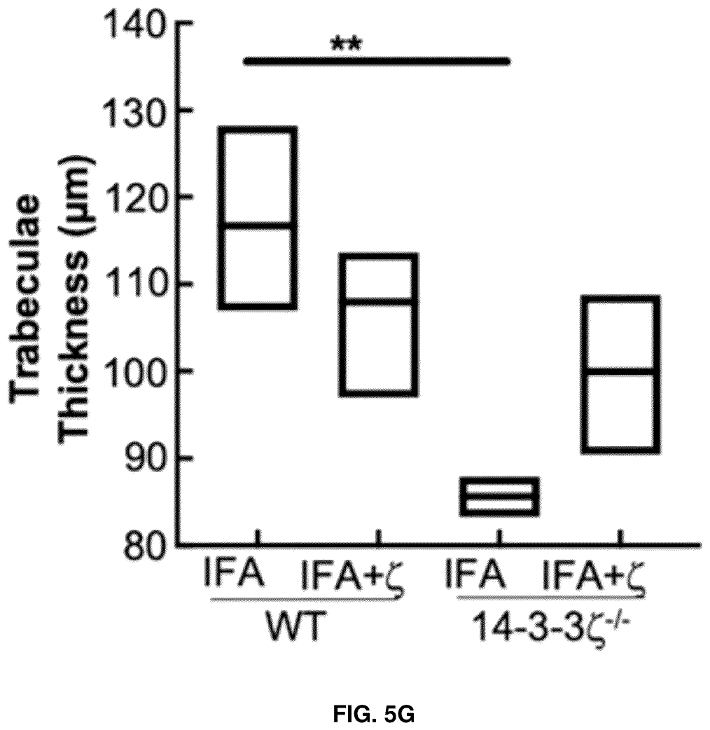

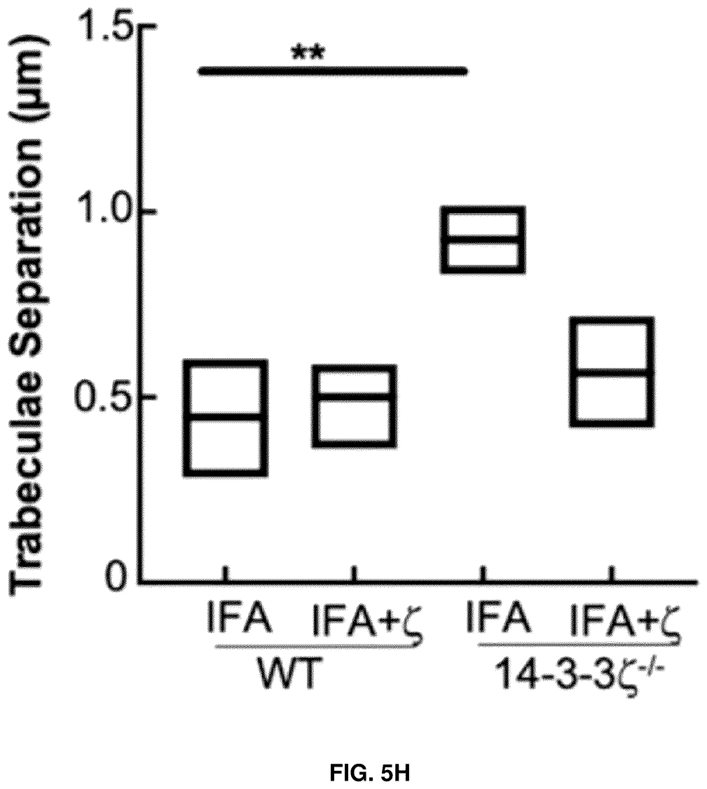

The findings of 14-3-3ζ immunization-based improvements in trabecular and cortical bone in the KO and WT animals led to an investigation of how 14-3-3ζ affects the overall bone quality. The μCT analysis of arthritic WT and KO bones revealed that the 14-3-3ζ loss resulted in the decreased cortical bone area, tissue mineral density, cortical bone thickness, trabecular bone/total bone volume ratio, connectivity density, and trabecular thickness (FIGS. 5A-5G). Marrow area and trabecular separation were increased in the proximal tibia (FIG. 5H). While most of these parameters were improved in the bones obtained from 14-3-3ζ-immunized WT and KO animals, the most significant improvements were observed in the parameters including tissue mineral density, trabecular bone/total bone volume ratio, and connectivity density (FIGS. 5C, 5E, 5F). Increased trabecular bone mass and trabeculae resulted in decreased trabecular separation by 14-3-3ζ immunization, further strengthening its direct impact on bone health (FIG. 5H).

### 14-3-3ζ Promotes Collagen Expression

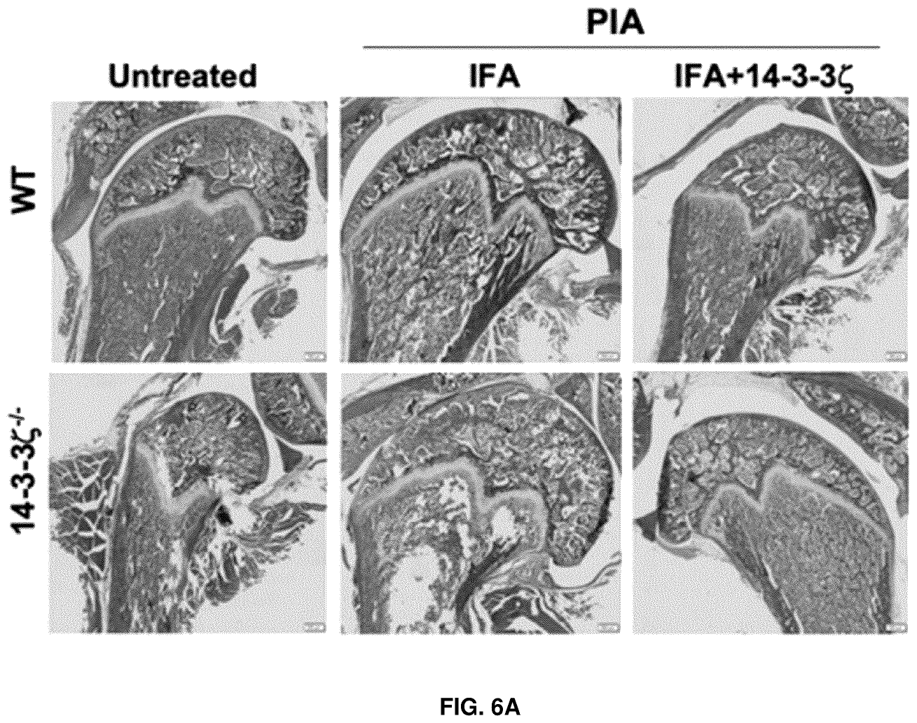

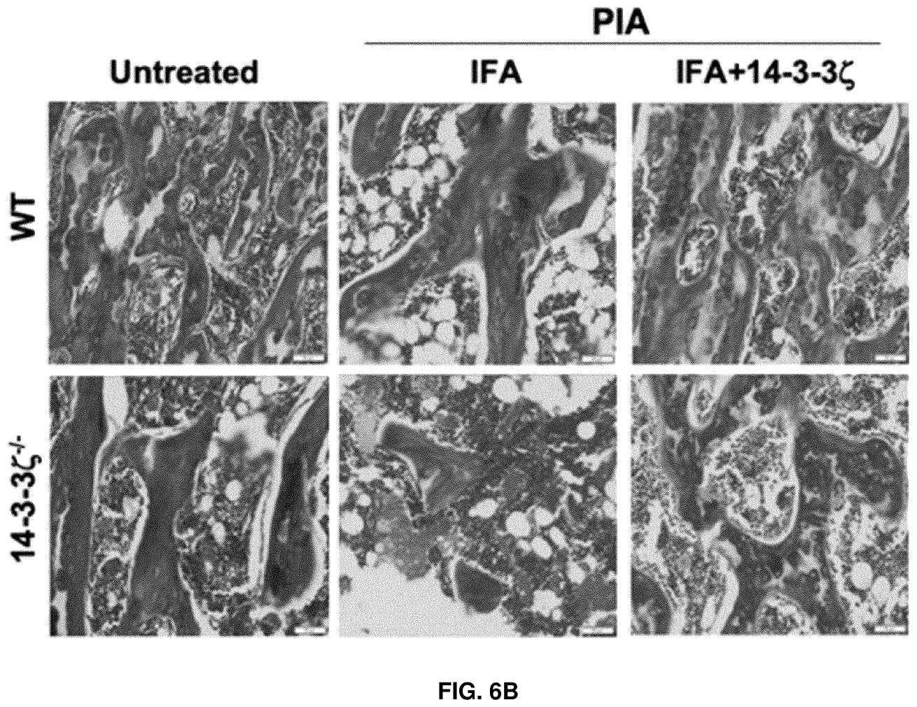

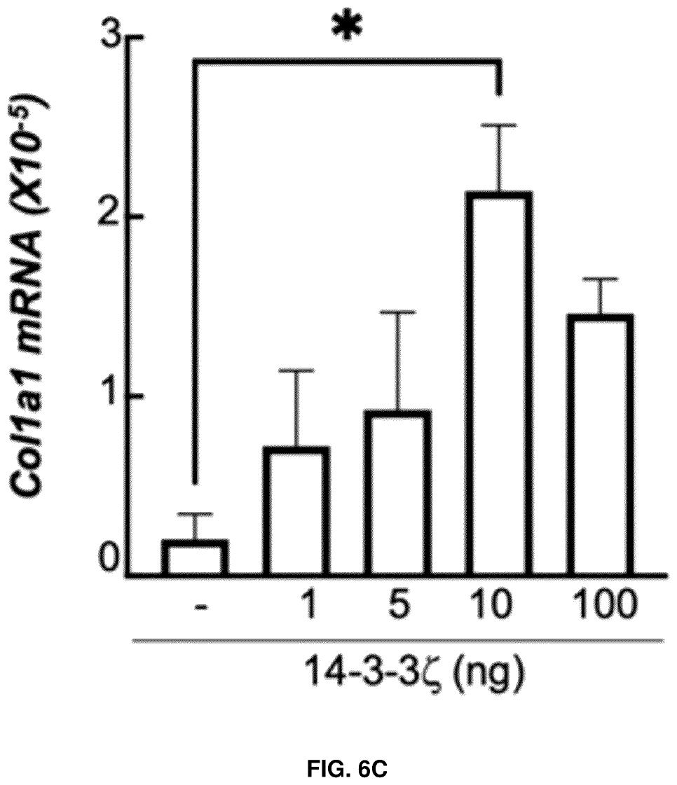

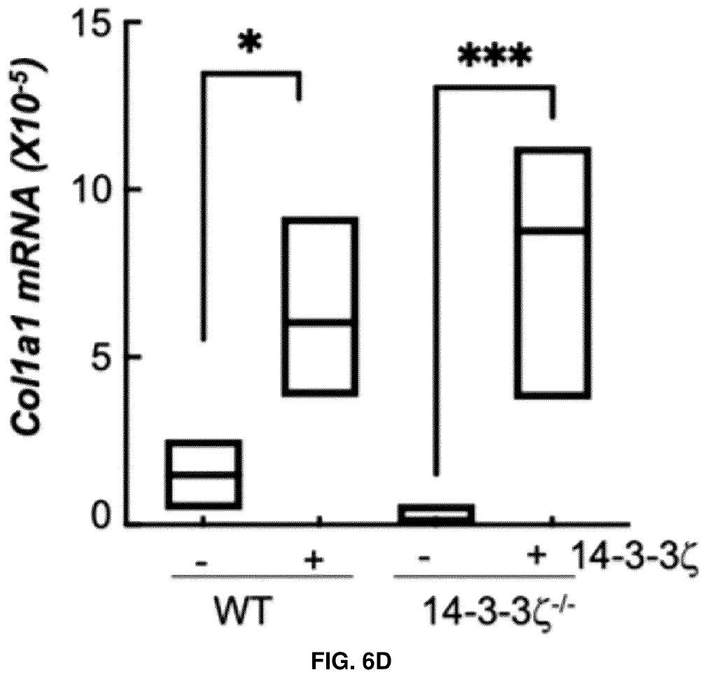

Following the observation of loss of tissue mineral density and trabecular bone, how 14-3-3ζ affects collagen level was examined Compared with WT, collagen staining in the naive KO animals was reduced. Induction of IA resulted in further loss of collagen levels in both WT and KO tibia (FIG. 6A). Collagen expression levels were restored upon 14-3-3ζ treatment (FIG. 6A). Similar to the overall bone, the collagen content of WT and KO trabecular bones was also significantly improved by 14-3-3ζ (FIG. 6B). To confirm the effect on collagen levels, primary rat bone marrow-derived mesenchymal cells that were cultured in osteoblast differentiating media in the presence of recombinant 14-3-3ζ protein purified from human embryonic kidney (HEK)293T cells were studied. In the presence of 14-3-3ζ. rat mesenchymal cells showed a dose-dependent increase in collagen 1 transcripts without significant impact on cell growth (FIG. 6C). Increased collagen 1 messenger RNA (mRNA) levels were also observed in mesenchymal cells isolated from 14-3-3ζ KO rats when treated with purified protein (FIG. 6D). These findings show that 14-3-3ζ promotes collagen induction in both in vivo and ex vivo models.

14-3-3ζ suppresses the IL-1β-signaling molecules

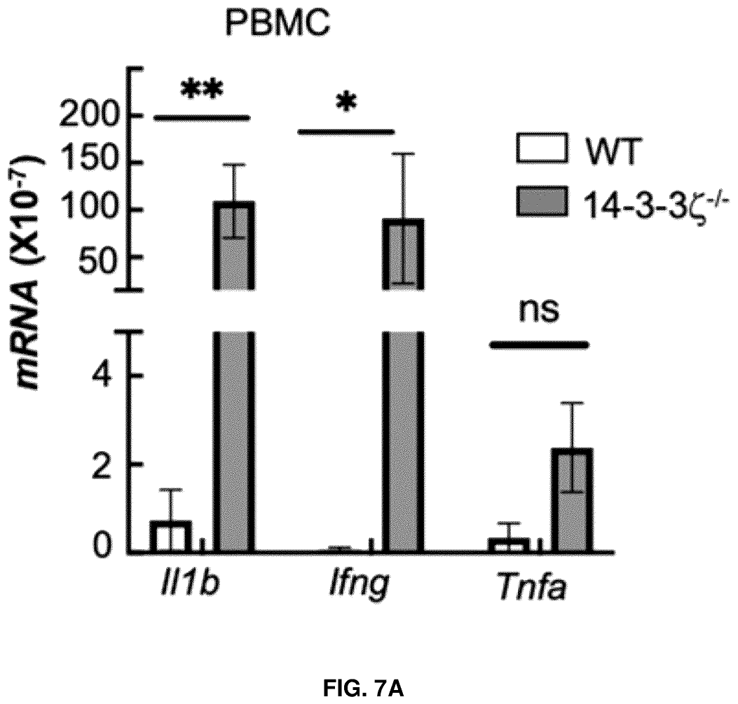

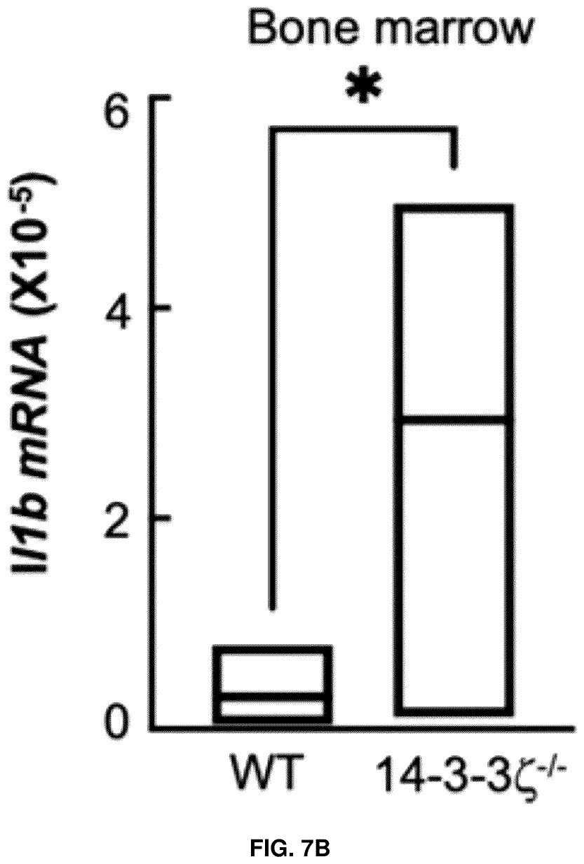

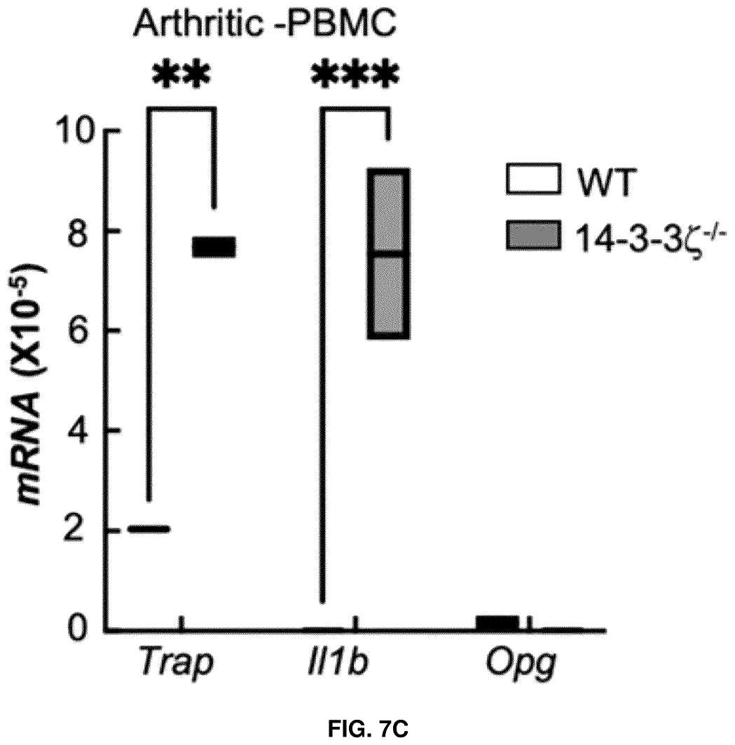

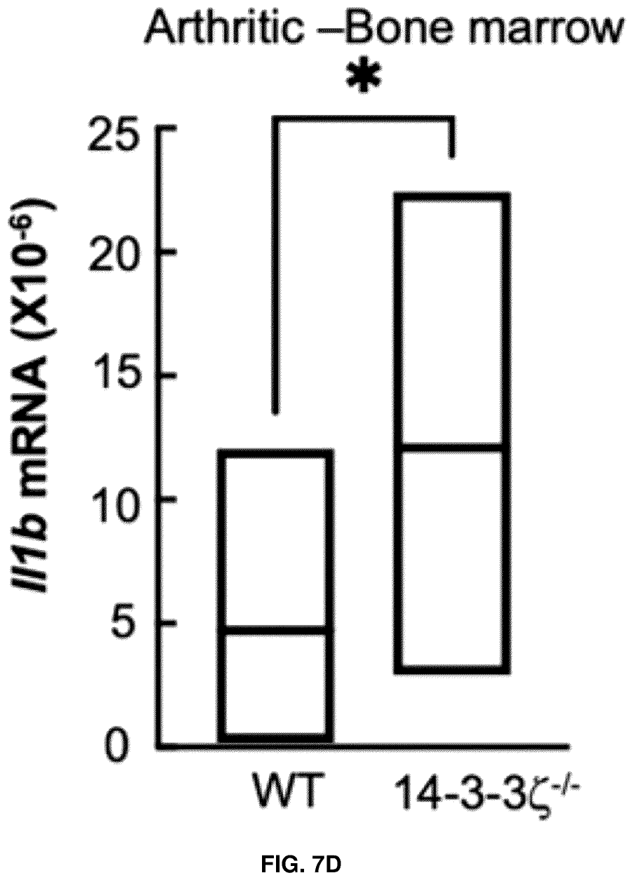

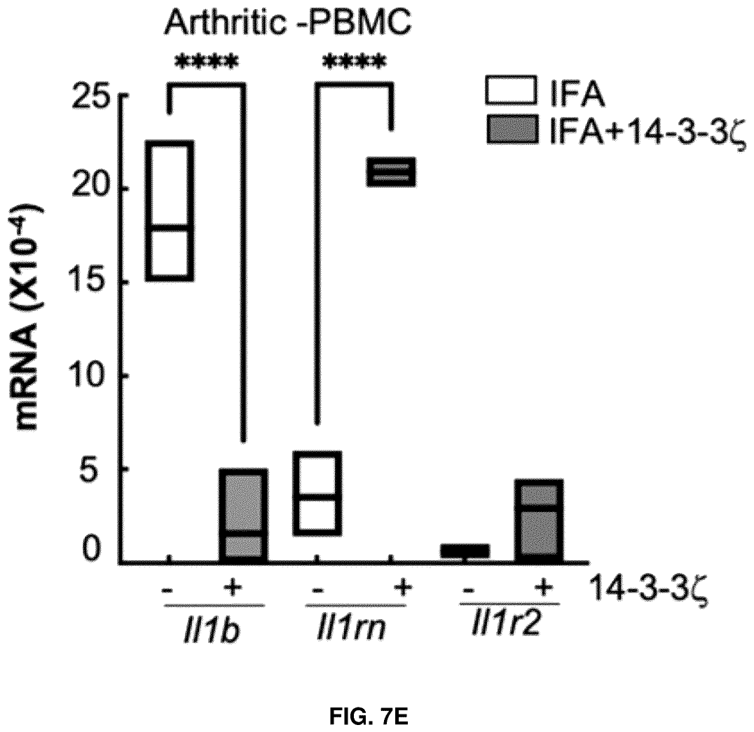

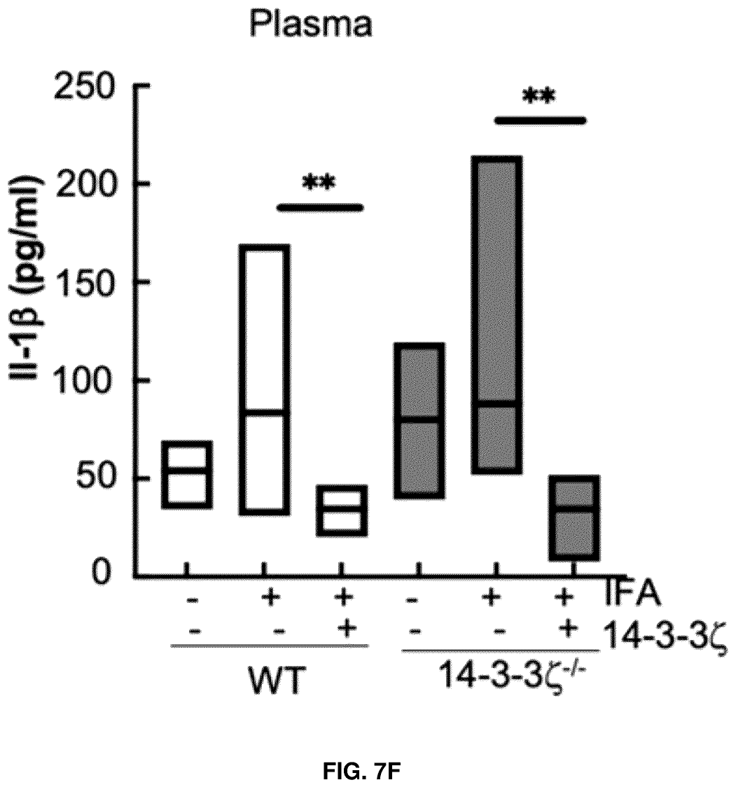

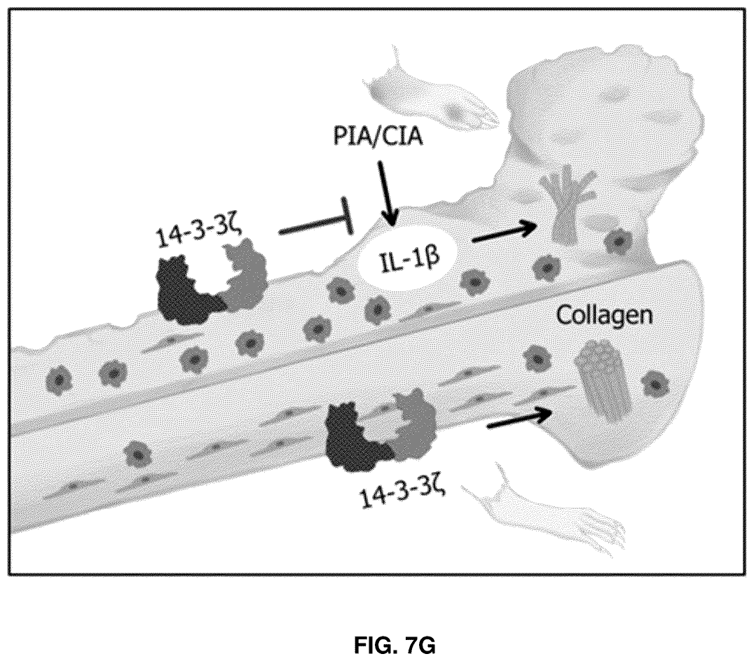

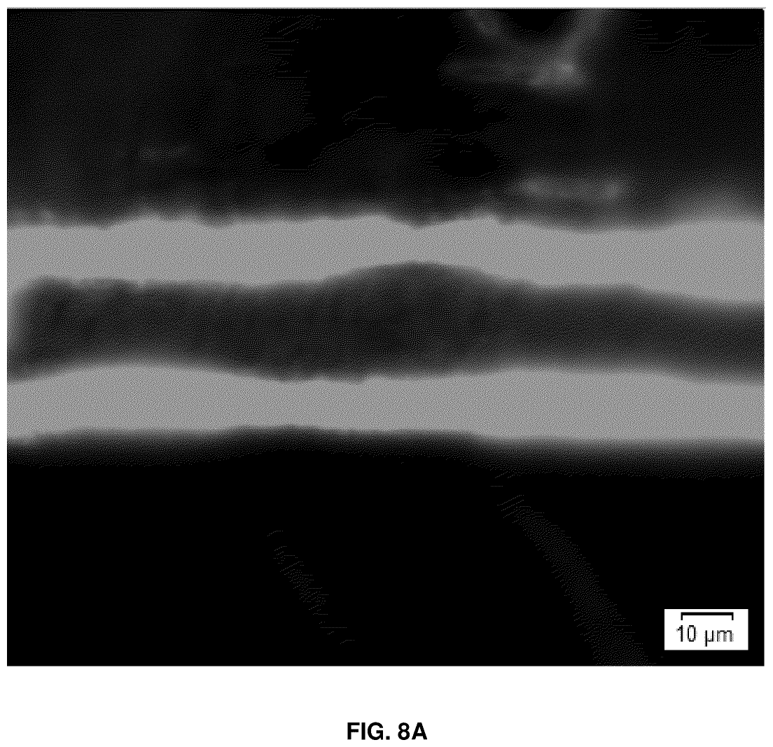

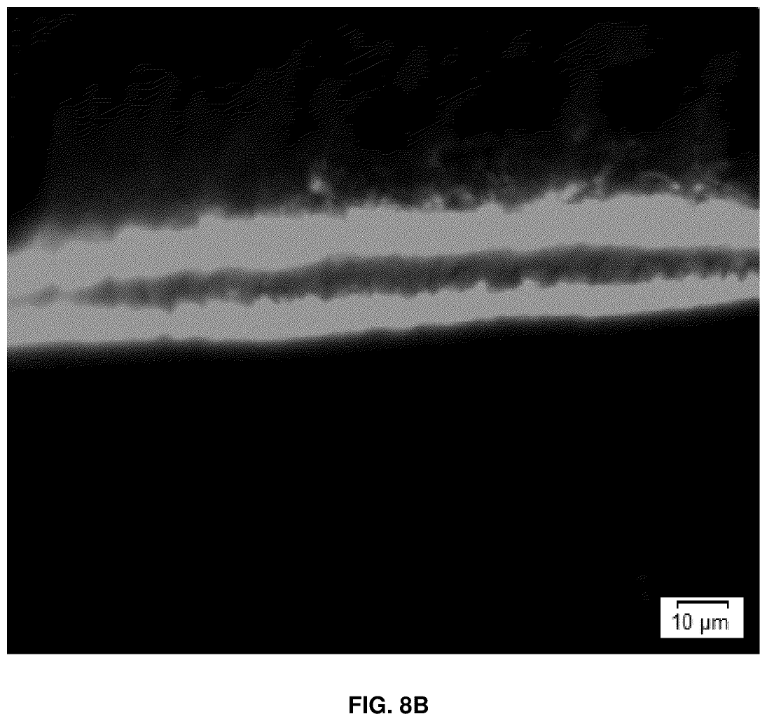

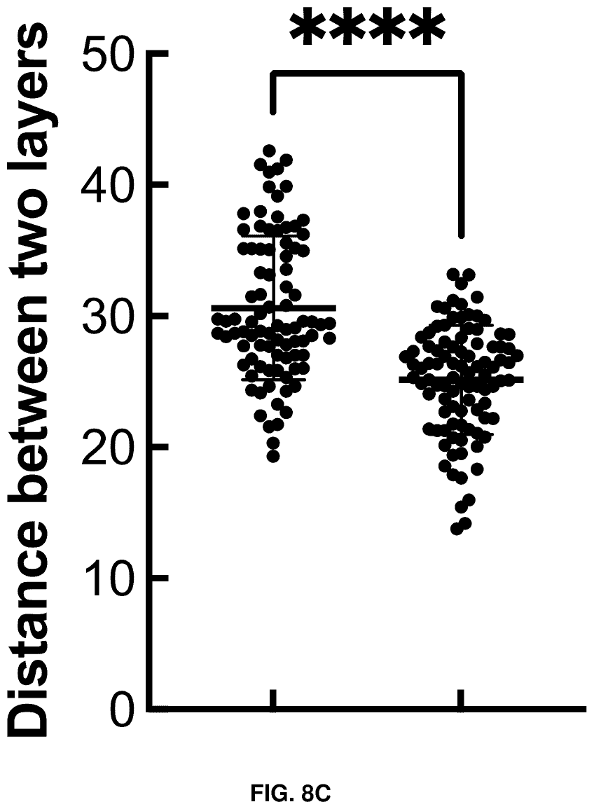

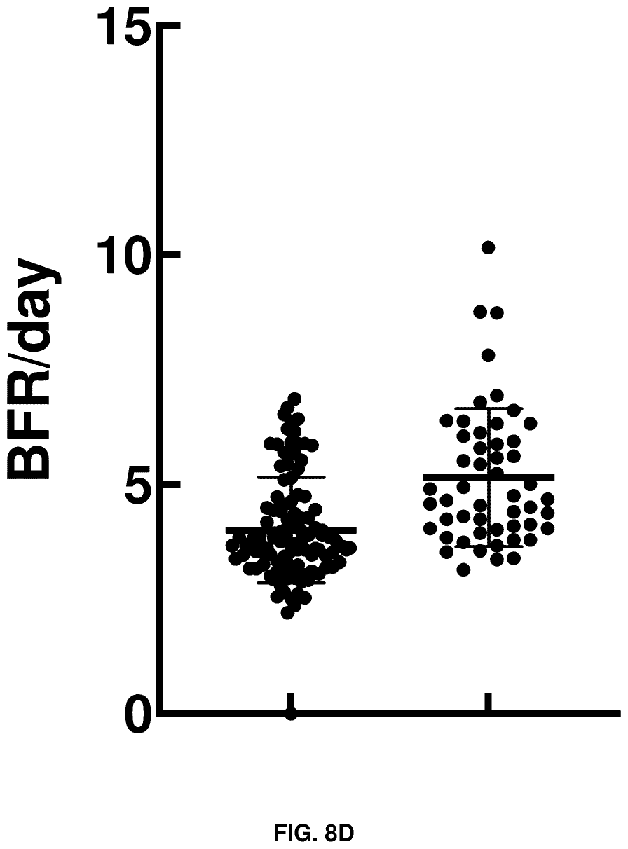

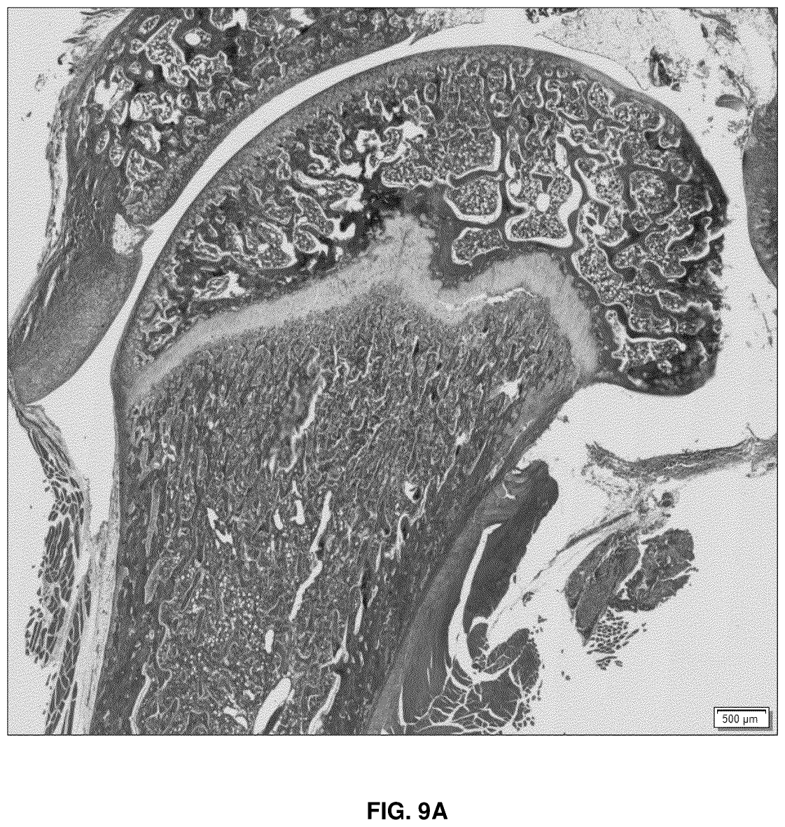

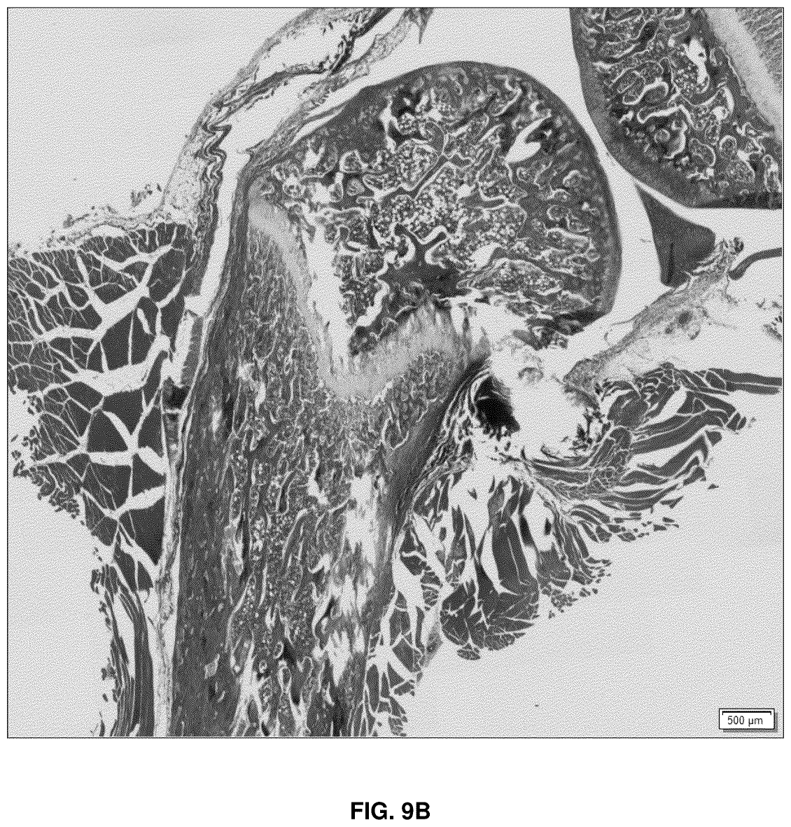

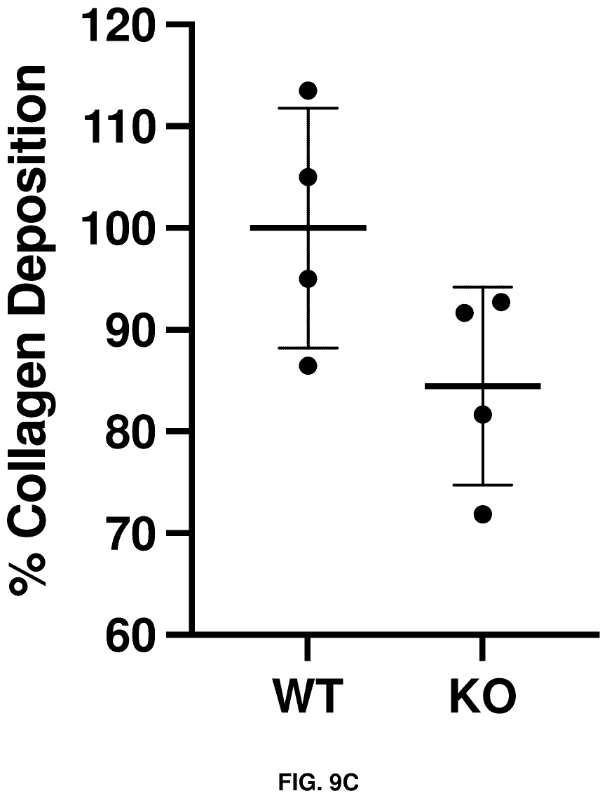

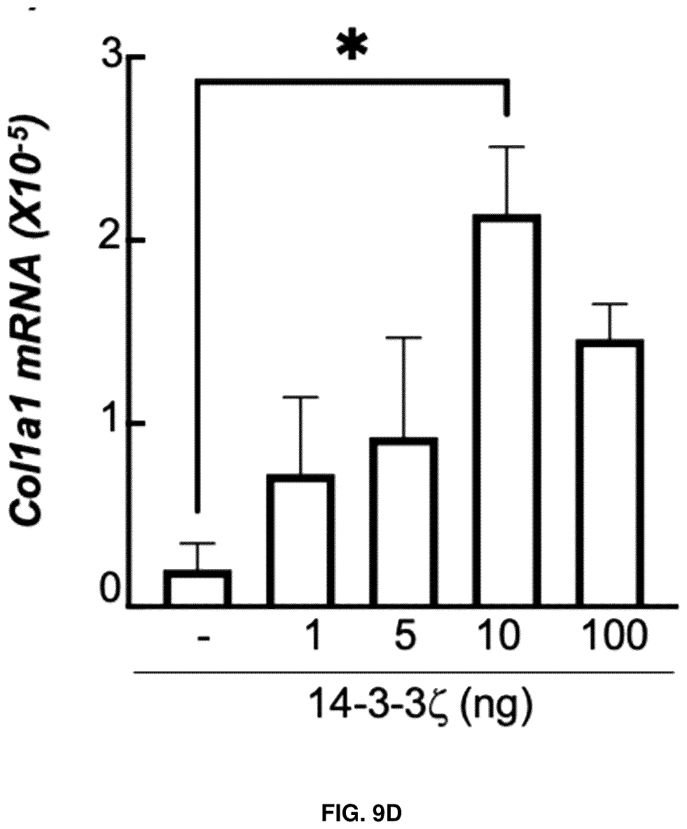

To investigate the molecular mechanism behind the IA-suppressive role of 14-3-3ζ, the immunological changes in naïve and arthritic 14-3-3ζ KO rats were examined Compared with WT rats, circulating peripheral blood mononuclear cells (PBMCs) of KO rats showed significantly higher Illb and Ifng but insignificant change in Tnfa (FIG. 7A). Expression of Ill7a or Il10 was undetectable in the KO animals Because IL-1β signaling plays a key role in RA pathogenesis, this cytokine was focused on in subsequent studies. As observed in PBMCs, Illb mRNA was also significantly elevated in the bone marrow of 14-3-3ζ KO rats (FIG. 7B). Next, whether a similar increase in Illb also occurred during IA was evaluated. Upon PIA induction, compared with WT, the Illb expression was higher in peripheral and bone marrow cells of 14-3-3ζ KO rats (FIGS. 7C, 7D). Furthermore, KO PBMCs also showed a significant increase in the tartrate-resistant acid phosphatase (Trap) expression, but not in osteoprotegerin (Opg), indicating that osteoclast activation may be responsible for the bone loss observed in the 14-3-3ζ KO rats (FIG. 7C).

Next, the effect of 14-3-3ζ immunization on Illb expression in the PIA model was examined PBMCs, collected from 14-3-3ζ-immunized animals after 1 wk of immunization, showed a significant reduction in Illb compared with animals treated with IFA only (FIG. 7E). In contrast, the IL-1 receptor antagonist (Illrn) expression level was elevated in the 14-3-3ζ-treated rats (FIG. 7E). Compared with the WT, the IL-1β protein was increased in naïve as well as pristane-treated KO rats (FIG. 7F). The 14-3-3ζ treatment led to reduced levels of circulating IL-1β protein levels in both WT and KO rats (FIG. 7F). Expression of other proinflammatory cytokines, including Cxcl1, Ifng, and Tnfa, was also reduced in the 14-3-3ζ-immunized animals

Collectively, these examples show that 14-3-3ζ causes immune suppression by interfering with the IL-1β pathway, as well as bone remodeling by promoting collagen synthesis, and functions as an endogenous suppressor of IA in vivo (FIG. 7G).

Effects of Wnt3a in 14-3-3zeta^{KO} MC3T3 Cells

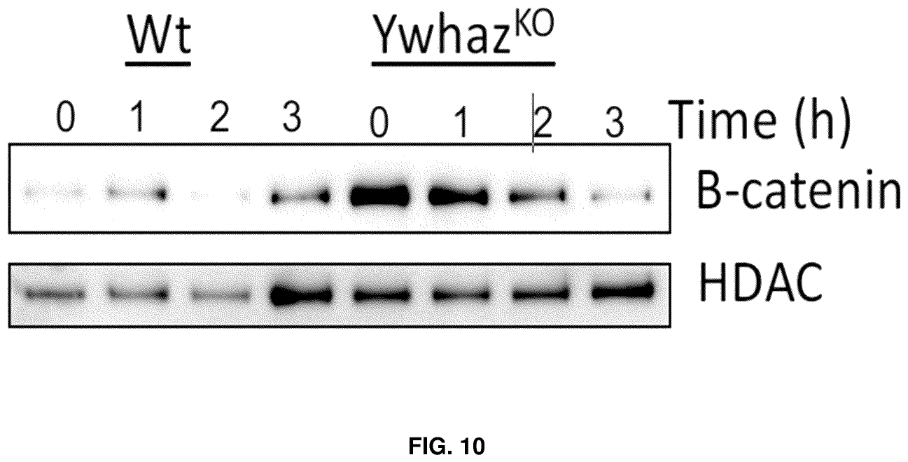

FIG. 10 shows that stimulation of 14-3-3zeta^{KO} (Ywhaz^{KO}) murine osteoblast (MC3T3) cells with Wnt3a reduces signal-induced β-catenin nuclear translocation as compared to wildtype (Wt) cells. This is important because activation of the Wnt signaling pathway by β-catenin nuclear translocation promotes osteoblast differentiation and bone formation, and inhibition of Wnt signaling leads to decreased bone formation and increased bone resorption.

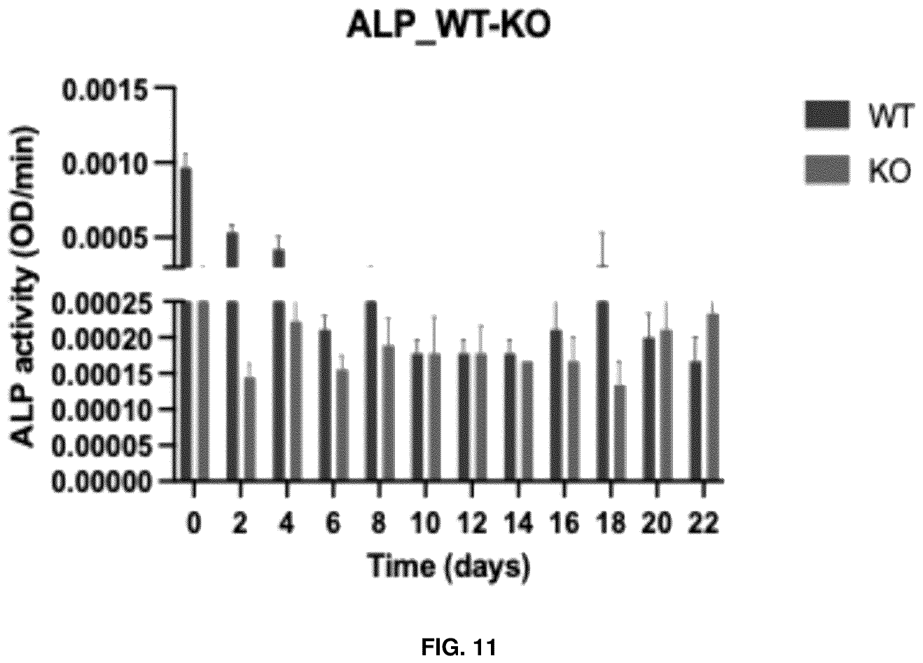

FIG. 11 shows that stimulation of 14-3-3zeta^{KO} (Ywhaz^{KO}) MC3T3 cells with Wnt3a results in reduced alkaline phosphatase (ALP) activity as compared to wildtype (Wt) cells. This is noteworthy because ALP plays a crucial role in osteoanabolism. ALP plays a role in the bone mineralization process by cleaving phosphate groups from matrix vesicles, with such phosphate groups then being released into the extracellular matrix where they react with calcium ions to form hydroxyapatite crystals, the main mineral component of bone. ALP also promotes osteoblast differentiation and participatation, which are essential for bone formation, and activates several signaling pathways involved in bone formation.

### Calcium Deposits

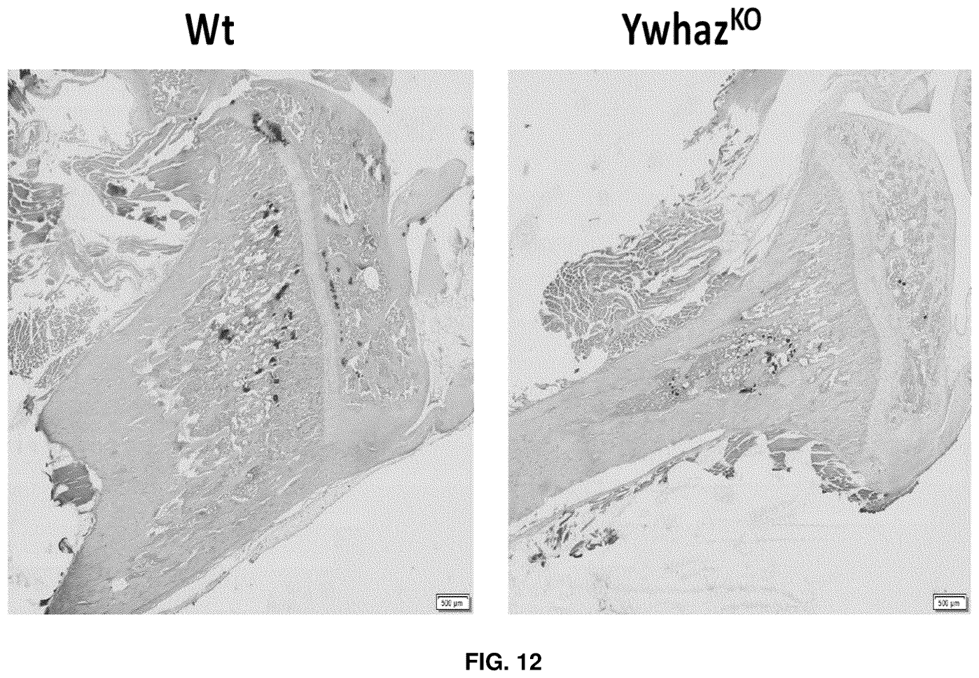

Bone sections from Wt and 14-3-3zeta^{KO} (Ywhaz^{KO}) animals were stained with alizarin. Alizarin is a red dye that binds specifically to calcium ions, forming a complex that can be visualized and quantified by light microscopy or spectrophotometry. Thus, alizarin staining is a method used to detect and quantify calcium deposition in cultured cells, particularly osteoblasts and chondrocytes. FIG. 12 shows the resulst, in which it is seen that 14-3-3zeta^{KO} (Ywhaz^{KO}) animals show reduced calcium deposits without 14-3-3zeta.

### RANKL—Stimulated Signal Transduction

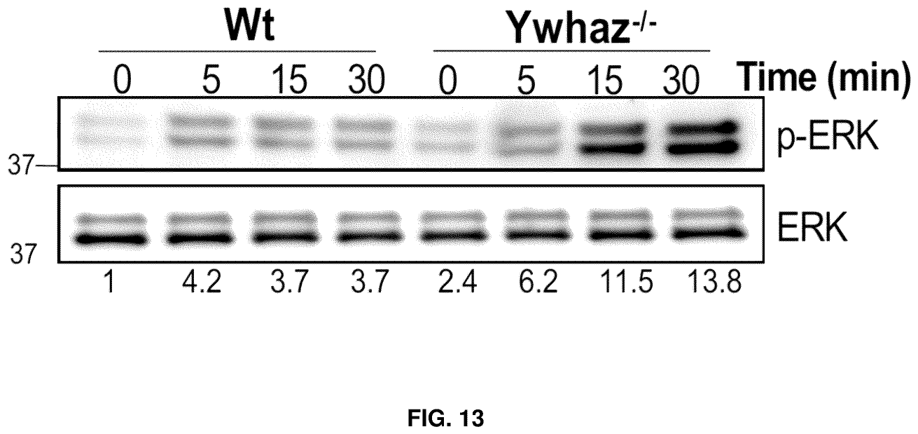

FIG. 13 shows that 14-3-3zeta suppresses RANKL-stimulated signal transduction in the murine macrophage (RAW 264.7) cells. Wt and 14-3-3zeta^{KO} (Ywhaz^{KO}) cells were treated with MCSF for min, followed by RANKL treatment for the indicated time to study the effect on phosphorylation of ERK. The results are shown in FIG. 13. The ability to suppress RANKL-stimulated signal transduction is important because inhibition of RANKL signaling can decrease the differentiation and activation of osteoclasts, which are responsible for bone resorption. As a result, inhibition of RANKL signaling can lead to decreased bone resorption and increased bone mass.

### Discussion

The 14-3-3ζ protein is a vital adaptor protein regulating several cellular processes including immune responses. These examples show that 14-3-3ζ has an arthritis-suppressive function in LEW rats. Global 14-3-3ζ KO rats show increased susceptibility to arthritis in both PIA and CIA models, providing strong evidence of the arthritis-suppressive role by an endogenous protein. The arthritic 14-3-3ζ KO animals showed increased bone surface damage with abnormal ectopic bone formation in the ankle and knee joints. Severe bone erosion in the growth plate and articular surfaces was observed in the knee joints and femoral heads of 14-3-3ζ KO rats. The 14-3-3ζ KO bones, including the femur and tibia (distal and proximal), showed a severe trabecular bone loss compared with the WT animals. Histological analyses confirmed the increased bone damage and synovial inflammation in 14-3-3ζ KO animals. The 14-3-3ζ immunization in both WT and 14-3-3ζ KO LEW rats protected animals from arthritis in both PIA and CIA models. Notably, the 14-3-3ζ immunization improved the collagen content, tissue mineral density, and trabecular bone volume. The improvement in joint inflammation was mirrored by a decrease in several proinflammatory cytokine productions, including Illb, Cxcl1, Ifng, and Tnfa. Time-course analysis showing up-regulation of film coupled with a decrease in Illb post-14-3-3ζ immunization may explain immune suppression caused by 14-3-3ζ. It is important to note that 14-3-3ζ inhibition of pyrindependent inflammasome activation has been previously reported. There were no visual or arthritic symptoms when animals were immunized with 14-3-3ζ in the absence of pristane. Similarly, increase in 14-3-3ζ antibody and IL-17A was not sufficient for induction of IA in Wistar rats that show resistance to arthritis. Therefore, it can be concluded that 14-3-3ζ has a suppressive effect on IA, which does not depend upon antibody level; instead, it requires active immunogenic function via suppression of IL-1α and promotion of collagen synthesis (FIG. 7G).

The PIA model is strongly affected by age but not by biological sex or housing environment. Pristane-induced cell death generates autoantigens recognized by major histocompatibility complex (MHC) class II-restricted arthritogenic T cells responsible for arthritis development. It has been shown that exogenous 14-3-3ζ promotes Thl and Th17 cell polarization in human PBMC and cytokine (IFN-γ and IL-17A) production. In the present examples, it was observed that 14-3-3ζ immunization resulted in robust antibody and significant IL-17A production but not IL-6. It was previously shown that 14-3-3ζ is required for IL-17A—stimulated IL-6 levels but not Cxcl-1 that may influence inflammation. It is also noteworthy that IFN-γ levels, but not IL-17A or IL-6, drive arthritis in the PIA rat model. The decrease in IL-1β, IFN-γ, and tumor necrosis factor (TNF)-α mRNA levels by 14-3-3ζ immunization may explain improved bone health and low arthritis scores. The 14-3-3ζ-mediated immune suppression did not involve IL-10; however, it was observed that 14-3-3ζ KO and IL-1R2 KO animals share several common features including increased arthritis susceptibility independent of T-cell and antibody responses. It is well documented that higher plasma and synovial IL-1α levels in RA contribute to the increased prostaglandin E2, matrix metalloproteases, and bone damage. IL-1α signaling requires IL-1R1, which is competitively inhibited by IL-1R2 and IL-1RA (IL1RN gene). Notably, IL-1RA inhibition is successfully used for treating RA.

A better understanding of IA's immune mechanisms has led to improved treatments, including cytokine blockers and other biologic therapies. However, the role of autoantigens (peptidyl arginine deiminase 4, glucose-6-phosphate isomerase, heat shock proteins, and heterogeneous nuclear ribonucleoprotein, etc.) in IA pathogenesis remains unclear. A few other autoantigens, including immunoglobulinbinding protein and DNAjp, have shown therapeutic potential in RA treatment by promoting anti-inflammatory responses. Unlike autoantigens, rheumatoid factor and anticitrullinated cyclic peptide antibodies associate with severe disease. In contrast, in these examples, it is shown that 14-3-3ζ antibodies decrease upon IA induction. Unlike other diseases, passive immunization with 14-3-3ζ antibodies did not affect IA pathogenesis. While the role of 14-3-3ζ antibodies remains debatable, their presence in healthy sera confirms the immunogenic nature of endogenous 14-3-3ζ in humans.

While 14-3-3ζ is predominantly intracellular, it does have an extracellular presence. In RA, activated B cells show reduced 14-3-3ζ peptide secretion. The basis of 14-3-3ζ antibody loss observed in arthritic animals in these examples can be explained by either a decreased level of antigenic peptide or loss of antigenicity. The presence of extracellular 14-3-3ζ in the sera of arthritic mice and urine of RA patients has previously been shown, and it is a primary secretory factor responsible for the resolution of arthritis in mice. Unlike the systemic effects observed in these examples, others have noticed local inflammation suppression upon increasing 14-3-3ζ using adenoviral constructs directly to the joints.

14-3-3ζ has a key role in arthritis and immune suppression. In summary, it is shown in these examples that 14-3-3ζ is an immunogen with a function of inflammatory arthritis suppression. These results indicate that the 14-3-3ζ participates in an endogenous hostprotective anti-arthritis immune mechanism. While these examples raise challenging questions related to 14-3-3cs role in other immune dysfunction and musculoskeletal abnormalities, 14-3-3ζ is a valuable tool in the prevention and treatment of IA.

### Methods

### Reagents

All common chemicals, including pristane, IFA, Luria-Broth media, ampicillin, isopropyl β-D-thiogalactoside (IPTG), and columns such as endotoxinremoving columns, were purchased from Fisher Scientific. The GST-14-3-3ζ construct was obtained from Addgene. The GST beads were obtained from Pierce Inc. The ELISA kits were purchased from R&D systems and PeproTech Inc.

### Purification of 14-3-3ζ Protein

The previously described protocol for 14-3-3ζ purification was utilized. Briefly, the BL-21 strain of Escherichia coli expressing GST-14-3-3ζ was grown and induced by 1 mM IPTG for 24 h. Bacteria were centrifuged and lysed by sonication. GST beads were used to pull down tagged 14-3-3ζ. which was eluted from the resin with 10 mM glutathione in 100 mM Tris·HCl (pH 8). Eluate was concentrated using an Amicon 30 K concentrator, then the GST tag was cleaved with thrombin (10 units/mg) for 2 h at 37 ° C. The cleaved tag was removed by incubating with fresh equilibrated GST resin for 1.5 h. The protein was then run through an endotoxin-removing spin column after incubating for at least 2 h. Both Coomassie staining and Western blot assessed protein purity after running sodium dodecyl sulfate—polyacrylamide gel electrophoresis. The his-tagged 14-3-3ζ was purified using Ni beads and cell lysates of HEK293T cells overexpressing recombinant protein. Imidazole-based elution followed by concentration and removal of imidazole was performed as per the recommended protocol.

### PIA

The 14-3-3ζ KO animals were generated as described before. Both WT and KO rats were maintained in the University of Toledo College of Medicine and Life Sciences vivarium and fed a standard diet. All animal experiments were conducted as per approved protocols by the Institutional Animal Care and Use Committee of the University of Toledo. Arthritis was induced in 8- to 10-wk-old male and female LEW rats by anesthetizing with 2% isoflurane in oxygen followed by intradermal injection of 200 μL pristine at the base of the tail. At days 1 and 7 post-pristane, either 100 μL IFA or a 1:1 ratio of IFA and purified 14-3-3ζ protein (1 mg/kg) was injected about 2 cm from the initial injection site. Animals were scored for arthritis twice every week unless specified otherwise. For scoring, a system of 0 to 80 with a max score of 20 possible for each limb was followed. Each joint of the foot was scored 0 (swelling absent) or 1 (swelling present). Swelling in the wrist, midforepaw, ankle, and midfoot was scored from 0 to 4. Body weight was measured once every week. At the end of the experiment, animals were killed, and body tissues, plasma, and synovial fluid were collected. For synovial fluid, 50 μL sterile phosphate buffered saline (PBS) was injected into the joint cavity. During the experiment, blood was collected at 15 d by saphenous vein bleeding into ethylenediaminetetraacetic acid (EDTA)-coated tubes.

### Plasma Infusion

Age 8- to 11-wk-old WT LEW rats, male and female, were injected with purified 14-3-3ζ protein (1 mg/kg) with IFA as an adjuvant. After 18 d, the rats were killed, and blood was collected in heparin-coated tubes. Half of the plasma obtained was incubated with 14-3-3ζ-bound GST beads (Pierce) overnight to remove 14-3-3ζ antibodies from the plasma. The depletion of 14-3-3ζ antibody in the plasma was confirmed by using ELISA. The 200 μL untreated or depleted plasma was intravenously injected through the tail vein.

### Mesenchymal Cell Culture

Cleaned bones from WT and 14-3-3ζ KO rats were collected and washed with sterile PBS. Marrow was collected by spinning it in a clean tube at 1,000 rpm for 5 min. The red blood cells cells were lysed using ammonium-chloride-potassium (ACK) lysis buffer, followed by plating in the Roswell Park Memorial Institute medium containing 15% fetal bovine serum, 0.2 mM ascorbic acid, and 10 mM b-glycerophosphate. After 2 d, nonadherent cells were removed, and the rest of the cells were cultured in the growth media containing dexamethasone until confluent.

### ELISA

Cytokines (IL-1β, IL-6, IL-17A, and TNF-a) were measured by using the commercial kits and protocols provided by the manufacturer (PeproTech Inc. and R&D Inc.). The 14-3-3ζ antibodies were measured using in-house ELISA. Briefly, the Immobilin 2B plates were coated with purified 14-3-3ζ at 50 ng/mL overnight at 4° C. Plates were blocked with 1% bovine serum albumin solution for 1 h at room temperature. Rat plasma samples were diluted in sterile PBS (7500× for IFA+14-3-3ζ-treated, 125× for IFA alone-treated). Diluted rat plasma in triplicate or synovial fluid were directly added to coated wells and incubated on a shaker for 2 h at room temperature (25° C.). After three washing steps in Tris-buffered saline (TBS)-Tween buffer for 5 min each, anti-rat-horseradish peroxidase diluted in TBS-Tween at 1:3,000 was added and incubated on a shaker for 1 h at room temperature. The plate was washed three times, 3,3′,5,5′-tetramethylbenzidine was added, and the development of color was observed. The reaction was stopped using 2N HCl, and the plate was read at 450 nm using a microplate reader. The absorbance of the control wells, including blank or no plasma controls, was used for subtraction. Commercial antibody at the 0- to 100-ng concentrations was used for the standard equation.

### Histology

Both knee and ankle joints were harvested from the killed rats and were cleaned of excess tissue. All samples were initially preserved in 10% neutral buffered formalin fixatives for 2 wk, followed by decalcification in acidified EDTA solution for 5 d. The bones were curetted by the Leica CM3050S (Leica Microsystems AG). All samples were sectioned in the vertical axis, cut at 5-μm thickness, and stained with hematoxylin and eosin or mason trichrome at the University of Toledo imaging core facility. All images were obtained with Olympus VS120-S6-W.

### μCT Imaging

Three-dimensional (3D) images of the proximal femur, knee joint, and ankle joint were acquired by μCT using the μCT 35 system (Scanco Medical AG) and using undivided hind leg specimens. Bone scans were performed with the X-ray source operating at 70-kVp and 40-μA energy settings and recording 500 projections/180° acquired at a 300-ms integration time using a 20-μm nominal resolution voxel for all bone locations. Scans encompassing regions of interest were segmented at an optimized lower threshold value of 170 units per mille scale (the equivalent of 2,055 Hounsfield units or linear attenuation coefficient [μ] of 1.36) and with a Gauss filter set to sigma 0.8 and support 1.0. These settings accommodated significant differences in radiodensity within regions of interest and provided an optimized view of the specimens by minimizing image erosion and image overrepresentation in KO and in WT bones, respectively. The 3D renderings of bone specimens were generated using μCT Ray version 4.0-4 software (Scanco Medical AG) with longitudinal sections recorded at ˜50% of the specimen depth.

RNA Isolation and qRT-PCR Analyses

Total RNA was isolated using TRIzol (Invitrogen), complementary DNA (cDNA) was prepared using the ImProm-II Reverse Transcription Kit (Promega), and the cDNA was analyzed using Radiant SYBR Green PCR mix (Alkali Scientific Inc.) in the Roche LightCycler 96 instrument and analyzed with the LightCycler 480 Software, version 1.5. The expression levels of the mRNAs were normalized to 18S ribosomal RNA. For the qRT-PCR analyses of the respective genes, the following primers were used:

Cxcl-1:
(SEQ ID NO: 2)
GGATTCACCTCAAGAACATCCAGA
(SEQ ID NO: 3)
CACCCTTCTACTAGCACAGTGGTTG
1l-10:
(SEQ ID NO: 4)
TGCCAAGCCTTGTCAGAAATGATCAAG
(SEQ ID NO: 5)
GTATCCAGAGGGTCTTCAGCTTCTCTC
Tnf-α:
(SEQ ID NO: 6)
ACC ACG CTC TTC TGT CTA CTG
(SEQ ID NO: 7)
CTT GGT GGT TTG CTA CGA C
Ifn-γ:
(SEQ ID NO: 8)
ATGAGTGCTACACGCCGCGTCTTGG
(SEQ ID NO: 9)
GAGTTCATTGACAGCTTTGTGCTGG
Il-1b:
(SEQ ID NO: 10)
GCAATGGTCGGGACATAGTT
(SEQ ID NO: 11)
AGACCTGACTTGGCAGAGGA
Il-1rn:
(SEQ ID NO: 12)
AAGACCTTCTACCTGAGGAACAACC
(SEQ ID NO: 13)
GCCCAAGAACACATTCCGAAAGTC
Il-r2:
(SEQ ID NO: 14)
CATTCAGACACCTCCAGCAGTTC
(SEQ ID NO: 15)
ACCCAGAGCGTATCATCCTTCAC
Il-17A:
(SEQ ID NO: 16)
CTTCACCCTGGACTCTGAGC
(SEQ ID NO: 17)
ATCTTCTCCACCCGGAAAGT

### Statistical Analysis

All experiments were performed at least thrice unless stated otherwise. Depending upon the number of sets for comparison, either an unpaired Student's t test or one-way ANOVA was used. P<0.05 was used for statistical significance.

Certain embodiments of the compositions and methods disclosed herein are defined in the above examples. It should be understood that these examples, while indicating particular embodiments of the invention, are given by way of illustration only. From the above discussion and these examples, one skilled in the art can ascertain the essential characteristics of this disclosure, and without departing from the spirit and scope thereof, can make various changes and modifications to adapt the compositions and methods described herein to various usages and conditions. Various changes may be made and equivalents may be substituted for elements thereof without departing from the essential scope of the disclosure. In addition, many modifications may be made to adapt a particular situation or material to the teachings of the disclosure without departing from the essential scope thereof.

What is claimed is:

1. A method of promoting bone growth in a subject, the method comprising administering to a subject an effective amount of a 14-3-3zeta protein or 14-3-3zeta mRNA to promote bone growth in the subject.

2. The method of claim 1, wherein the effective amount is about 1 mg/kg.

3. The method of claim 1, wherein the 14-3-3zeta protein or 14-3-3zeta mRNA is in a composition with an adjuvant.

4. The method of claim 3, wherein the adjuvant comprises incomplete Freund's adjuvant (IFA).

5. The method of claim 1, comprising administering two doses of the 14-3-3zeta protein or 14-3-3zeta mRNA to the subject.

6. The method of claim 5, wherein the two doses promotes bone growth in the subject for at least 45 days.

7. The method of claim 1, wherein the 14-3-3zeta protein is administered to the subject through a dermal patch, an ointment, or an injectable vaccine.

8. The method of claim 1, wherein the subject has an elevated risk of a bone fracture, has inflammatory arthritis, is at least 55 years old, is a post-menopausal woman, has osteoporosis, has diabetes, is trauma-affected, or is post-operative.

9. A method of preventing bone damage in a subject, the method comprising administering to the subject an effective amount of a 14-3-3zeta protein or 14-3-3zeta mRNA to prevent bone damage in the subject.

10. The method of claim 9, wherein the effective amount is about 1 mg/kg.

11. The method of claim 9, wherein the 14-3-3zeta protein or 14-3-3zeta mRNA is in a composition with an adjuvant.

12. The method of claim 9, comprising administering two doses of the 14-3-3zeta protein or 14-3-3zeta mRNA to the subject.

13. The method of claim 9, wherein the 14-3-3zeta protein is administered to the subject through a dermal patch, an ointment, or an injectable vaccine.

14. The method of claim 9, wherein the subject has an elevated risk of a bone fracture, has inflammatory arthritis, is at least 55 years old, is a post-menopausal woman, has osteoporosis, has diabetes, is trauma-affected, or is post-operative.

15. A method of regulating collagen production in a subject, the method comprising administering to the subject an effective amount of a 14-3-3zeta protein or 14-3-3zeta mRNA to regulate collagen production in the subject.

16. The method of claim 15, wherein the effective amount is about 1 mg/kg.

17. The method of claim 15, wherein the 14-3-3zeta protein or 14-3-3zeta mRNA is in a composition with an adjuvant.

18. The method of claim 15, comprising administering two doses of the 14-3-3zeta protein or 14-3-3zeta mRNA to the subject.

19. The method of claim 15, wherein the 14-3-3zeta protein is administered to the subject through a dermal patch, an ointment, or an injectable vaccine.

20. The method of claim 15, wherein the subject has an elevated risk of a bone fracture, has inflammatory arthritis, is at least 55 years old, is a post-menopausal woman, has osteoporosis, has diabetes, is trauma-affected, or is post-operative.


SEQUENCE LISTING (17 sequences)

SEQ ID NO: 1 - protein, 245 aa, Homo sapiens
MDKNELVQKAKLAEQAERYDDMAACMKSVTEQGAELSNEERNLLSVAYKNVVGARRSSWRVVSSIEQKTEGAEKKQQMAREYREKIETELRDICNDVLSLLEKFLIPNASQPESKVFYLKMKGDYYRYLAEVAAGDDKKGIVDQSQQAYQEAFEISKKEMQPTHPIRLGLALNFSVFYYEILNSPEKACSLAKTAFDEAIAELDTLSEESYKDSTLIMQLLRDNLTLWTSDTQGDEAEAGEGGEN

SEQ ID NO: 2 - DNA, 24 bp, synthetic construct
  source 1..24: mol_type = other DNA
ggattcacctcaagaacatccaga

SEQ ID NO: 3 - DNA, 25 bp, synthetic construct
  source 1..25: mol_type = other DNA
cacccttctactagcacagtggttg

SEQ ID NO: 4 - DNA, 27 bp, synthetic construct
  source 1..27: mol_type = other DNA
tgccaagccttgtcagaaatgatcaag

SEQ ID NO: 5 - DNA, 27 bp, synthetic construct
  source 1..27: mol_type = other DNA
gtatccagagggtcttcagcttctctc

SEQ ID NO: 6 - DNA, 21 bp, synthetic construct
  source 1..21: mol_type = other DNA
accacgctcttctgtctactg

SEQ ID NO: 7 - DNA, 19 bp, synthetic construct
  source 1..19: mol_type = other DNA
cttggtggtttgctacgac

SEQ ID NO: 8 - DNA, 25 bp, synthetic construct
  source 1..25: mol_type = other DNA
atgagtgctacacgccgcgtcttgg

SEQ ID NO: 9 - DNA, 25 bp, synthetic construct
  source 1..25: mol_type = other DNA
gagttcattgacagctttgtgctgg

SEQ ID NO: 10 - DNA, 20 bp, synthetic construct
  source 1..20: mol_type = other DNA
gcaatggtcgggacatagtt

SEQ ID NO: 11 - DNA, 20 bp, synthetic construct
  source 1..20: mol_type = other DNA
agacctgacttggcagagga

SEQ ID NO: 12 - DNA, 25 bp, synthetic construct
  source 1..25: mol_type = other DNA
aagaccttctacctgaggaacaacc

SEQ ID NO: 13 - DNA, 24 bp, synthetic construct
  source 1..24: mol_type = other DNA
gcccaagaacacattccgaaagtc

SEQ ID NO: 14 - DNA, 23 bp, synthetic construct
  source 1..23: mol_type = other DNA
cattcagacacctccagcagttc

SEQ ID NO: 15 - DNA, 23 bp, synthetic construct
  source 1..23: mol_type = other DNA
acccagagcgtatcatccttcac

SEQ ID NO: 16 - DNA, 20 bp, synthetic construct
  source 1..20: mol_type = other DNA
cttcaccctggactctgagc

SEQ ID NO: 17 - DNA, 20 bp, synthetic construct
  source 1..20: mol_type = other DNA
atcttctccacccggaaagt